SEQUENCE LISTING (17 sequences)

SEQ ID NO: 1 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 2 - DNA, 191 bp, synthetic construct
  source 1..191: mol_type = other DNA
ccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtc

SEQ ID NO: 3 - DNA, 1145 bp, synthetic construct
  source 1..1145: mol_type = other DNA
acaatcatagctcactgcagccttgagctcccaggctcaagtgatcctcccgcctcagcctcctgagtagctgagatcacaggcatgcactaccacactcggctcacatttgacatcctctaaagcatatataaaatgtgaagaaaactttcacaatttgcatccctttgtaatatgtaacagaaataaaattctcttttaaaatctatcaacaataggcaaggcacggtggctcacgcctgtcgtctcagcactttgtgaggcccaggcgggcagatcgtttgagcctagaagttcaagaccaccctggccaacatagcgaaaccccctttctacaaaaaatacaaaaactagctgggtgtggtggtgcacacctgtagtcccagctacttggaaggctgaaatgggaagactgcttgagcccgggagggggaagttgcagtaagccaggaccacaccactgcactccagcctgggcaacagagtgagactctgtctcaaacaaacaaataaatgaggcgggtggatcacgaggtcagtagatcgagaccatcctggctaacacggtgaaacccgtctctactaaaaaaaaaaaaaaaatacaaaaaattagccaggcatggtggcgggcgcctgtagtcccagttactcgggaggctgaggcaggagaatggcgtgaaaccgggaggcagagcttgcagtgagccgagatcgcaccactgccctccagcctgggcgacagagcgagactccgtctcaatcaatcaatcaatcaataaaatctattaacaatatttattgtgcacttaacaggaacatgccctgtccaaaaaaactttacagggcttaactcattttatccttaccacaatcctatgaagtaggaacttttataaaacgcattttataaacaaggcacagagaggttaattaacttgccctctggtcacacagctaggaagtgggcagagtacagatttacacaaggcatccgtctcctggccccacatacccaactgctgtaaacccataccggcggccaagcagcctcaatttgtgcatgcacccacttcccagcaagacagcagctcccaagttcctcctgtttagaattttagaagcggcgggccaccaggctgctctag

SEQ ID NO: 4 - DNA, 220 bp, synthetic construct
  source 1..220: mol_type = other DNA
agtctcccttgggtcaggggtcctggttgcactccgtgctttgcacaaagcaggctctccatttttgttaaatgcacgaatagtgctaagctgggaagttcttcctgaggtctaacctctagctgctcccccacagaagagtgcctgcggccagtggccaccaggggtcgccgcagcacccagcgctggagggcggagcgggcggcagacccggagcagc

SEQ ID NO: 5 - DNA, 165 bp, synthetic construct
  source 1..165: mol_type = other DNA
atgtggaccctcggccgcagagctgttgctggacttcttgcctctccatctccagctcaggcccagacactgaccagagtgcctagacctgctgaactggcccctctgtgtggcagaagaggcctgagaaccgacatcgacgccacatgcacacctagaagggcc

SEQ ID NO: 6 - DNA, 152 bp, synthetic construct
  source 1..152: mol_type = other DNA
gtaagtatccgcgccgggaacagccgcgggccgcacgccgcgggccgcacgccgcacgcctgcgcagggaggcgccgcaatatataaattatgcattaatgggttataattcactgaaaaatagtaacgtacttcttaactttggctttcag

SEQ ID NO: 7 - DNA, 474 bp, synthetic construct
  source 1..474: mol_type = other DNA
agcagcaatcagcggggcctgaatcaaatctggaacgtgaagaaacagagcgtgtacctgatgaacctgagaaagagcggcaccctgggacaccctggaagcctggatgagacaacctacgagagactggccgaggaaaccctggattccctggccgagttcttcgaggacctggccgataagccctacaccttcgaggattacgacgtgtcctttggcagcggcgtgctgacagtgaaactcggcggagatctgggcacctacgtgatcaacaagcagacccctaacaaacagatctggctgagcagccctagcagcggccccaagagatatgattggaccggcaagaactgggtgtacagccacgatggcgtgtccctgcacgaactgctggctgccgaactgacaaaggccctgaaaacaaagctggacctgtccagcctggcctactctggcaaggacgcctgatgatga

SEQ ID NO: 8 - DNA, 1809 bp, synthetic construct
  source 1..1809: mol_type = other DNA
tcgagtgccccagccccgttttaaggacattaaaagctatcaggccaagaccccagcttcattatgcagctgaggtctgttttttgttgttgttgttgtttattttttttattcctgcttttgaggacagttgggctatgtgtcacagctctgtagaaagaatgtgttgcctcctaccttgcccccaagttctgatttttaatttctatggaagattttttggattgtcggatttcctccctcacatgataccccttatcttttataatgtcttatgcctatacctgaatataacaacctttaaaaaagcaaaataataagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacatgcatctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttgg

SEQ ID NO: 9 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 10 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 11 - DNA, 124 bp, synthetic construct
  source 1..124: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggcca

SEQ ID NO: 12 - DNA, 50 bp, synthetic construct
  source 1..50: mol_type = other DNA
taataaaagatccttattttcattggatctgtgtgttggttttttgtgtg

SEQ ID NO: 13 - DNA, 268 bp, synthetic construct
  source 1..268: mol_type = other DNA
tccccctgcccccacagctcctctcctgtgccttgtttcccagccatgcgttctcctctataaatacccgctctggtatttggggttggcagctgttgctgcctgggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattg

SEQ ID NO: 14 - protein, 55 aa, synthetic construct
MWTLGRRAVAGLLASPSPAQAQTLTRVPRPAELAPLCGRRGLRTDIDATCTPRRA

SEQ ID NO: 15 - protein, 155 aa, synthetic construct
SSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 16 - DNA, 1337 bp, synthetic construct
  source 1..1337: mol_type = other DNA
gatatcggtaccatttagcatcctaagtatgtaaacatgactctcttcacgattcacaaagtggctttggaagaactttagtaccttcccatcttctctgccatggaaagtgtacacaactgacattttcttttttttaagacagtatcttgctatgatggccgggctggaatgctgtggctattcacaggcacaatcatagctcactgcagccttgagctcccaggctcaagtgatcctcccgcctcagcctcctgagtagctgagatcacaggcatgcactaccacactcggctcacatttgacatcctctaaagcatatataaaatgtgaagaaaactttcacaatttgcatccctttgtaatatgtaacagaaataaaattctcttttaaaatctatcaacaataggcaaggcacggtggctcacgcctgtcgtctcagcactttgtgaggcccaggcgggcagatcgtttgagcctagaagttcaagaccaccctggccaacatagcgaaaccccctttctacaaaaaatacaaaaactagctgggtgtggtggtgcacacctgtagtcccagctacttggaaggctgaaatgggaagactgcttgagcccgggagggggaagttgcagtaagccaggaccacaccactgcactccagcctgggcaacagagtgagactctgtctcaaacaaacaaataaatgaggcgggtggatcacgaggtcagtagatcgagaccatcctggctaacacggtgaaacccgtctctactaaaaaaaaaaaaaaaatacaaaaaattagccaggcatggtggcgggcgcctgtagtcccagttactcgggaggctgaggcaggagaatggcgtgaaaccgggaggcagagcttgcagtgagccgagatcgcaccactgccctccagcctgggcgacagagcgagactccgtctcaatcaatcaatcaatcaataaaatctattaacaatatttattgtgcacttaacaggaacatgccctgtccaaaaaaactttacagggcttaactcattttatccttaccacaatcctatgaagtaggaacttttataaaacgcattttataaacaaggcacagagaggttaattaacttgccctctggtcacacagctaggaagtgggcagagtacagatttacacaaggcatccgtctcctggccccacatacccaactgctgtaaacccataccggcggccaagcagcctcaatttgtgcatgcacccacttcccagcaagacagcagctcccaagttcctcctgtttagaattttagaagcggcgggccaccaggctgctctag

SEQ ID NO: 17 - DNA, 755 bp, synthetic construct
  source 1..755: mol_type = other DNA
ttggaaggctgaaatgggaagactgcttgagcccgggagggggaagttgcagtaagccaggaccacaccactgcactccagcctgggcaacagagtgagactctgtctcaaacaaacaaataaatgaggcgggtggatcacgaggtcagtagatcgagaccatcctggctaacacggtgaaacccgtctctactaaaaaaaaaaaaaaaatacaaaaaattagccaggcatggtggcgggcgcctgtagtcccagttactcgggaggctgaggcaggagaatggcgtgaaaccgggaggcagagcttgcagtgagccgagatcgcaccactgccctccagcctgggcgacagagcgagactccgtctcaatcaatcaatcaatcaataaaatctattaacaatatttattgtgcacttaacaggaacatgccctgtccaaaaaaactttacagggcttaactcattttatccttaccacaatcctatgaagtaggaacttttataaaacgcattttataaacaaggcacagagaggttaatttacttgccctctggtcacacagctaggaagtgggcagagtacagatttacacaaggcatccgtctcctggccccacatacccaactgctgtaaacccataccggcggccaagcagcctcaatttgtgcatgcacccacttcccagcaagacagcagctcccaagttcctcctgtttagaattttagaagcggcgggccaccaggctgctctag

(19) United States
(12) Patent Application Publication
(10) Pub. No.: US 2025/0312489 A1
(43) Pub. Date: Oct. 9, 2025

(54) NOVEL METHODS AND COMPOSITION OF AAV VECTORS FOR THE TREATMENT OF FRIEDREICH'S ATAXIA

(71) Applicant: University of Florida Research Foundation, Incorporated, Gainesville, FL (US)

(72) Inventors: Barry John Byrne, Gainesville, FL (US); Manuela Corti, Gainesville, FL (US)

(73) Assignee: University of Florida Research Foundation, Incorporated, Gainesville, FL (US)

(21) Appl. No.: 19/173,488

(22) Filed: Apr. 8, 2025

Related U.S. Application Data
(60) Provisional application No. 63/631,298, filed on Apr. 8, 2024.

(51) Int. Cl. A61K 48/00; A61K 31/436; A61K 38/17; A61P 25/28; C07K 16/28; C12N 15/86

(52) U.S. Cl. CPC A61K 48/005; A61K 31/436; A61K 38/1709; A61P 25/28; C07K 16/2875; C07K 16/2887; C12N 15/86; C12N 2750/14121; C12N 2750/14143; C12N 2830/50

(57) ABSTRACT

The present disclosure relates to nucleic acids, recombinant adeno-associated virus (rAAV) particles, compositions, and methods related to gene therapy for Friedreich's ataxia (FRDA). In some embodiments, the nucleic acids disclosed herein provide an optimal level of frataxin expression in a cell or a subject.

### CROSS-REFERENCE TO RELATED APPLICATION

This application claims the benefit under 35 U.S.C. § 119 (e) of U.S. Provisional Application No. 63/631,298, filed on Apr. 8, 2024, entitled “NOVEL METHODS AND COMPOSITION OF AAV VECTORS FOR THE TREATMENT OF FRIEDRIECH'S ATAXIA,” the entire disclosure of which is incorporated by reference herein.

### GOVERNMENT SUPPORT

This invention was made with government support under grant number U01 NS116752 awarded by the National Institutes of Health. The government has certain rights in the invention.

### REFERENCE TO AN ELECTRONIC SEQUENCE LISTING

The contents of the electronic sequence listing (U120270147US01-SEQ-AXW.xml; Size: 23,161 bytes; and Date of Creation: Apr. 4, 2025) are herein incorporated by reference in its entirety.

### BACKGROUND

Friedreich's ataxia is a genetic disease that causes damage to the heart and nervous system, resulting in degeneration of the cerebellar and sensory neurons, as well as fatal cardiomyopathy. Friedreich's ataxia is caused by a mutation in the FXN gene that is due to an expansion of an intronic GAA repeat, which leads to reduced expression of the mitochondrial protein frataxin. There is currently only one approved therapy for Friedreich's ataxia.

### BRIEF SUMMARY

Aspects of the disclosure relate to nucleic acids, recombinant adeno-associated virus (rAAV) particles, compositions, and methods related to gene therapy for Friedreich's ataxia (FRDA).

Aspects of the present disclosure provide recombinant adeno associated virus (rAAV) vectors (e.g., nucleic acids or recombinant genomes) comprising frataxin expression constructs. In some embodiments, an rAAV vector comprises a frataxin expression construct flanked by AAV ITRs. In some embodiments, a frataxin expression construct comprises a frataxin control region. In some embodiments, a frataxin expression construct comprises a frataxin exon 1 noncoding and/or coding region. In some embodiments, a frataxin expression construct comprises a frataxin intron. In some embodiments, a frataxin expression construct comprises frataxin exons 2-5. In some embodiments, a frataxin expression construct comprises a combination of two or more elements selected from: frataxin control region, exon 1 noncoding and coding region, frataxin intron, and frataxin exons 2-5. In some embodiments, a frataxin expression construct comprises a frataxin control region, exon 1 noncoding and coding region, frataxin intron, and frataxin exons 2-5.

In some embodiments, the frataxin control region comprises a sequence that is at least 90, 95, 99, or 99.5 percent identical to any one of SEQ ID NOs: 3, 16, and 17.

In some embodiments, the exon 1 noncoding region comprises a sequence that is at least 90, 95, or 99 percent identical to SEQ ID NO: 4.

In some embodiments, the exon 1 coding region comprises a sequence that is at least 90, 95, or 99 percent identical to SEQ ID NO: 5.

In some embodiments, the frataxin intron comprises a sequence that is at least 90, 95, or 99 percent identical to SEQ ID NO: 6.

In some embodiments, the frataxin exons 2-5 comprise a sequence that is at least 90, 95, or 99 percent identical to SEQ ID NO: 7.

In some embodiments, the frataxin expression construct (e.g., nucleic acid or recombinant genome) comprises, from 5′ to 3′, the frataxin control region, the exon 1 noncoding region, the exon 1 coding region, the frataxin intron, and the frataxin exons 2-5. In some embodiments, the rAAV vector further comprises an additional element, wherein the additional element comprises an enhancer sequence, a polyA-encoding sequence, inverted terminal repeats (ITRs), or a 3′ untranslated region (UTR) sequence.

In some embodiments, the rAAV vector comprises the sequences shown in Table 1.

In some embodiments, the rAAV vector comprises the sequences shown in Table 2.

In some embodiments, the rAAV vector comprises the sequences shown in Table 3.

In some embodiments, the rAAV vector comprises the sequences shown in Table 4.

In some embodiments, the rAAV vector comprises the sequences shown in Table 5.

Aspects of the present disclosure provide an rAAV particle comprising an rAAV vector of the present disclosure.

Aspects of the present disclosure provide a composition comprising any of the rAAV vectors of the present disclosure. In some embodiments, the composition comprises a pharmaceutically acceptable carrier.

Aspects of the present disclosure provide a method of treating a subject in need thereof, the method comprising administering an effective amount of any of the rAAV vectors of the present disclosure or any of the compositions of this disclosure.

Aspects of the present disclosure provide a method of increasing frataxin expression in a cell, the method comprising administering an effective amount of any of the rAAV vectors of the present disclosure or any of the compositions of the present disclosure.

Aspects of the present disclosure provide a method of treating Friedreich's Ataxia in a subject, the method comprising administering an effective amount of any of the rAAV vectors of the present disclosure or any of the compositions of the present disclosure.

In some embodiments, the subject is human. In some embodiments, the subject has, is suspected of having, or is at risk for FRDA.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a table of the plasmid log numbers and the corresponding plasmid names.

FIGS. 2A-2D show diagrams of four non-limiting examples of self-complementary AAV vectors that express frataxin (FXN). FIG. 2A shows a self-complementary AAV vector that expresses frataxin under the control of the endogenous frataxin promoter (FXN Control). FIG. 2B shows a self-complementary AAV vector that expresses frataxin under the control of the endogenous frataxin promoter with a desmin enhancer (DesEnh) element to increase expression in the heart (AAV9-DE7-hFXN). FIG. 2C shows a schematic of the B757 vector, comprising a coFXN2 gene under the control of a chicken beta-actin (CBA) promoter. FIG. 2D shows a schematic of the B800 vector, which expresses a P4-frataxin construct.

FIG. 3 is a table showing an overview of the experimental design (plasmid, cells, DNA concentration, and harvest timeline) for the promoter comparison in vitro transfection study design.

FIG. 4 shows the frataxin expression observed in in vitro transfected 293T cells. Abcam hFXN ELSA data on protein extracted cell pellet was performed using TGEK-50 extraction buffer. The amount of protein loaded was normalized to the pierce BCA quantification. Two values are shown for each construct: the left bar shows frataxin expression observed with 1 μg of construct DNA transfected into 293T cells; the right bar shows frataxin expression observed with 5 μg of construct DNA transfected into 293T cells.

FIG. 5 shows western blot results from a C2C12 in vitro study comparing expression using different promoters and enhancer elements. The western blot was performed using C2C12 cell pellets harvested 7 days after differentiation with Myo-D. B757 was loaded at a 1:2 dilution all other samples loaded at 1:1 dilution. A commercial mouse monoclonal primary antibody to frataxin was used.

FIG. 6 shows the vector packaging and titer of CO63 construct. The titer without underlining is the estimated titer based on the stain free gel analysis. The underlined titer is the ddPCR titer including the positive control for all ddPCR runs.

FIG. 7 shows the vector packaging and titer of CO96 construct. The titer without underlining is the estimated titer based on the stain free gel analysis. The underlined titer is the ddPCR titer.

FIG. 8 is a table showing the experimental design of the promoter comparison in vivo mouse study. The B757 dose (underlined) is the Vector core qPCR titer that was considered low dose after ddPCR; The dose was back calculated to the actual dose after IV injection. The B757 administration was repeated at the 5E+13 dose (groups 2 and 5).

FIG. 9A shows quantification of the number of vector genome copies per ug gDNA transfected in neonatal and adult mouse tissues.

FIG. 9B shows expression of frataxin (pg FXN/ug total protein) expressed in the heart using the B800 (P4-FXN) construct and the B757 (using the CBA promoter) vectors in heart tissue.

FIG. 10 shows a western blot measuring the frataxin peptide produced by different constructs in neonatal mouse heart tissue.

FIG. 11-shows quantification of the FXN expression in neonatal heart tissue from mice dosed with 5E+13 vector genomes/kg body weight from the same experiment as FIG. 10. FIG. 11 shows picograms of expressed frataxin (pg FXN) per milligram of total protein (mg protein) for vehicle treated cells, cells dosed with vector C063 (AAV9-E7-hFXN), vector B908 (Mut5-P4-hFXN), vector C096 (AAV9-DE7-hFXN), and B757 (AAV9-CBA-hFXN) vectors, (left to right) as shown on the x-axis.

FIG. 12 shows the numerical quantification of frataxin expression from the experiment described for FIG. 11.

FIG. 13 is a table showing the experimental design of the C096 (AAV9-D7-hFXN) immunosuppression adult study. The aim of this study is to identify the most effective immunosuppression regimen that leads to higher expression after a single or repeat administration of rAAV.

FIGS. 14A-14D show ELISA measurements of frataxin expression in the heart (FIG. 14A), brain (FIG. 14B), cerebellum (FIG. 14C), and spinal cord (FIG. 14D) of subjects treated with the indicated immunosuppressive treatment(s).

FIG. 15 shows the experimental design of a dose escalation experiment using the C151 vector (equivalent to C096 (AAV9-DE7-hFXN)).

FIGS. 16A-16C show measurements of frataxin expression in the heart (FIG. 16A), brain (FIG. 16B), and cerebellum (FIG. 16C) of mice treated according to the table of FIG. 15, as a dose-escalation study.

FIG. 17 shows the experimental design of a non-human primate (NHP) study using the new vector.

FIG. 18 shows expression of human frataxin (pg FXN/ug total protein) in NHP heart tissue with rapamycin and rituximab, no immunosuppression, or vehicle treatment.

FIG. 19A-19D shows blood marker readouts over time from the NHP study described herein. FIG. 19A shows alkaline phosphatase levels over time (stars indicate additional Gr 3 points). FIG. 19B shows alanine aminotransferase (ALT) levels over time. FIG. 19C shows platelet count over time. FIG. 19D shows a key identifying each group.

FIG. 20 shows the experimental design of a phenotypic mouse model using AAV9-DE7-hFXN.

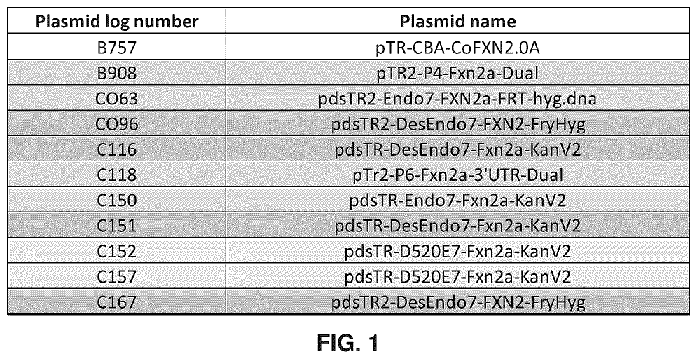

### DETAILED DESCRIPTION

The present disclosure provides recombinant adeno-associated virus (rAAV) vectors encoding human frataxin which utilize regulatory sequences from the native frataxin gene locus. In some embodiments, the rAAV vectors utilize segments of intron 1 which impact regulation of frataxin mRNA. In some embodiments, the rAAV vectors contemplated herein produce optimal levels of frataxin expression. In some embodiments, the rAAV vectors contemplated herein avoid frataxin overexpression. In some embodiments, the rAAV vectors comprise nucleic acid sequences encoding frataxin. The present disclosure also provides rAAV particles comprising the rAAV vectors disclosed herein. The present disclosure also provides cells comprising the rAAV vectors disclosed herein. The present disclosure also provides methods of making and using the rAAV vectors and cells comprising the same. In some embodiments, the rAAV vectors, or rAAV particles comprising the same are used to treat a subject. In some embodiments, the subject has or is suspected of having Friedreich's ataxia.

Friedrich's ataxia (FRDA) is an autosomal recessive disorder caused by a trinucleotide repeat expansion (TNR) in the first intron of the frataxin (FXN) gene, on chromosome 9q12-13. The repeat expansion leads to a deficiency in the mitochondrial protein frataxin (FXN). FRDA affects 1 in 50,000 people worldwide and is characterized by progressive sensory neural degeneration, resulting in ataxia, sensory loss, muscle weakness, and hypertrophic cardiomyopathy. Symptoms generally present at puberty and patients have a shorter than normal life expectancy reaching 40-50 years of age.

Frataxin is a highly conserved, 210 amino acid (˜17 kDa) protein encoded in the nucleus. While frataxin's specific function remains unclear, homozygous deletions are embryonically lethal. Evidence suggests frataxin is involved in iron metabolism, iron storage, iron-sulfur cluster (ISC) formation, and protection against reactive oxygen species (ROS). Dysregulation of FXN leads to iron accumulation in the mitochondria and insufficient iron in the cytoplasm. Excess mitochondrial iron increases the incidence of iron-catalyzed reduction of hydrogen peroxide generating toxic ROS. The increase in ROS disrupts iron homeostasis in the mitochondria and affects the ISC aconitase, a major component of cellular respiration.

There is only one FDA approved therapy for FRDA: Skyclarys® (Reata Pharmaceuticals) (omaveloxolone). Omaveloxolone administration may be associated with side effects, such as elevated liver enzymes, headache, nausea, abdominal pain, fatigue, diarrhea, and musculoskeletal pain. Therefore, there is a need for additional treatment options for FRDA, especially treatment options with fewer or less severe side effects compared to omaveloxolone.

The major neurological symptoms of FRDA include muscle weakness and ataxia, a loss of balance and coordination. FRDA mostly affects the spinal cord and the peripheral nerves that connect the spinal cord to the body's muscles and sensory organs. FRDA affects the function of the cerebellum and also the musculature of the heart. There is a high prevalence of diabetes in FRDA patients as well. FXN deficiency in pancreatic islet cells causes diabetes (Ristow, M, et al., J Clin Invest. 112 (4): 527-534, 2003).

rAAV Vectors

In some aspects, the present disclosure provides recombinant adeno associated virus (rAAV) vectors that are useful for achieving optimal levels of frataxin expression in a cell. In some embodiments, an rAAV vector comprises a rAAV genome (recombinant genome). In some embodiments, an rAAV vector comprises a plasmid comprising an rAAV genomic sequence (e.g., for use in manufacturing rAAV particles).

In some aspects, the present disclosure contemplates rAAV vectors comprising a frataxin expression construct comprising frataxin control region, exon 1 noncoding and coding region, frataxin intron 1, and frataxin exons 2-5. In some embodiments, frataxin exons 1-5 comprise wildtype frataxin exon 1-5 sequences. In some embodiments, frataxin exons 2-5 comprise wildtype frataxin exon 2-5 sequences. In some embodiments, frataxin exons 2-5 are provided without their natural introns. In some embodiments, the only intron provided is a frataxin intron having SEQ ID NO: 6, or a variant thereof. As described below, in some embodiments, an rAAV vector comprises one or more additional elements, such as inverted terminal repeats (ITRs), enhancers, or 3′ untranslated regions (UTRs). In some embodiments, an rAAV vector comprises a frataxin expression construct flanked by ITRs.

### Frataxin Expression Constructs

In some aspects, the present disclosure contemplates rAAV vectors for increasing frataxin expression in a cell or a subject. In some embodiments, an rAAV vector associated with the disclosure comprises a frataxin expression construct. In some embodiments, rAAV vectors comprising a frataxin expression construct associate with the disclosure produce a therapeutically optimal level of frataxin expression.

In some embodiments, the frataxin expression construct comprises a promoter. In some embodiments, the promoter is a tissue-specific (e.g., cardiac-specific, muscle-specific, or neuron-specific) promoter. In some embodiments, the promoter is a native frataxin promoter. In some embodiments, the promoter is a chicken beta-actin promoter. In some embodiments, the promoter is a desmin promoter. In some embodiments, the promoter is a synthetic promoter.

In some embodiments, the frataxin expression construct comprises an enhancer sequence (also called an enhancer). In some embodiments, the enhancer sequence is or comprises a desmin enhancer sequence. In some embodiments, the enhancer sequence is or comprises an alpha-myosin enhancer sequence. In some embodiments, the enhancer sequence is or comprises an alpha-myosin heavy chain enhancer sequence.

In some embodiments, the frataxin expression construct comprises a native frataxin promoter and an enhancer sequence. In some embodiments, the frataxin expression construct comprises a native frataxin promoter and a desmin enhancer sequence.

In some embodiments, the frataxin expression construct comprises a frataxin control region sequence. In some embodiments, the frataxin control region sequence is of varying length. In some embodiments, the frataxin control region is a long frataxin control region (e.g., SEQ ID NO: 16). In some embodiments, the frataxin control region is a mid-length frataxin control region (e.g., SEQ ID NO: 3). In some embodiments, the frataxin control region is a short frataxin control region (e.g., SEQ ID NO: 17). In some embodiments, the frataxin control region sequence comprises a sequence at least 70%, 75%, 80%, 85%, 90%, 95%, 99%, 99.5%, or 99.9% identical to any one of SEQ ID NOs: 3, 16, and 17. In some embodiments, the frataxin control region sequence comprises a sequence at least 70%, 75%, 80%, 85%, 90%, 95%, 99%, 99.5%, or 99.9% identical to SEQ ID NO: 3. In some embodiments, the frataxin control region sequence comprises a sequence at least 70%, 75%, 80%, 85%, 90%, 95%, 99%, 99.5%, or 99.9% identical to SEQ ID NO: 16. In some embodiments, the frataxin control region sequence comprises a sequence at least 70%, 75%, 80%, 85%, 90%, 95%, 99%, 99.5%, or 99.9% identical to SEQ ID NO: 17. In some embodiments, the frataxin control region sequence comprises 1-5, 5-10, 15-20, 20-25, 25-30, 35-40, 45-50, 55-60, 65-70 or more amino acid substitutions relative to any one of SEQ ID NOs: 3, 16, and 17. In some embodiments, the frataxin control region sequence comprises 1-5, 5-10, 15-20, 20-25, 25-30, 35-40, 45-50, 55-60, 65-70 or more amino acid substitutions relative to SEQ ID NO: 3. In some embodiments, the frataxin control region sequence comprises 1-5, 5-10, 15-20, 20-25, 25-30, 35-40, 45-50, 55-60, 65-70 or more amino acid substitutions relative to SEQ ID NO: 16. In some embodiments, the frataxin control region sequence comprises 1-5, 5-10, 15-20, 20-25, 25-30, 35-40, 45-50, 55-60, 65-70 or more amino acid substitutions relative to SEQ ID NO: 17. In some embodiments, the frataxin control region sequence comprises the sequence of any one of SEQ ID NOs: 3, 16, and 17. In some embodiments, the frataxin control region sequence comprises the sequence of SEQ ID NO: 3. In some embodiments, the frataxin control region sequence comprises the sequence of SEQ ID NO: 16. In some embodiments, the frataxin control region sequence comprises the sequence of SEQ ID NO: 17. In some embodiments, the frataxin expression construct comprises a frataxin exon 1 non-coding sequence. In some embodiments, the frataxin exon 1 non-coding sequence comprises a sequence at least 70%, 75%, 80%, 85%, 90%, 95%, 99%, or 99.5% identical to SEQ ID NO: 4. In some embodiments, the frataxin exon 1 non-coding sequence comprises 1-5, 5-10, 15-20, 20-25, 25-30, 35-40, 45-50, 55-60, 65-70 or more amino acid substitutions relative to SEQ ID NO: 4. In some embodiments, the frataxin exon 1 non-coding sequence comprises the sequence of SEQ ID NO: 4. In some embodiments, the frataxin expression construct comprises a frataxin exon 1 coding sequence. In some embodiments, the frataxin exon 1 coding sequence comprises a sequence at least 70%, 75%, 80%, 85%, 90%, 95%, or 99% identical to SEQ ID NO: 5. In some embodiments, the frataxin exon 1 coding sequence comprises 1-5, 5-10, 15-20, 20-25, 25-30, 35-40, 45-50, 55-60, 65-70 or more amino acid substitutions relative to SEQ ID NO: 5. In some embodiments, the frataxin exon 1 coding sequence comprises the sequence of SEQ ID NO: 5. In some embodiments, the frataxin expression construct comprises a frataxin exon 1 coding sequence encoding the amino acid sequence of SEQ ID NO: 14. In some embodiments, the frataxin expression construct comprises a frataxin intron 1 sequence. In some embodiments, the frataxin intron 1 sequence comprises a sequence at least 70%, 75%, 80%, 85%, 90%, 95%, or 99% identical to SEQ ID NO:6. In some embodiments, the frataxin intron 1 sequence comprises the sequence of SEQ ID NO: 6. In some embodiments, the frataxin expression construct comprises a frataxin exon 2-5 sequence. In some embodiments, the frataxin exon 2-5 sequence comprises a sequence at least 70%, 75%, 80%, 85%, 90%, 95%, 99%, or 99.5% identical to SEQ ID NO: 7. In some embodiments, the frataxin exon 2-5 sequence comprises 1-5, 5-10, 15-20, 20-25, 25-30, 35-40, 45-50, 55-60, 65-70 or more amino acid substitutions relative to SEQ ID NO: 7. In some embodiments, the frataxin exon 2-5 sequence comprises the sequence of SEQ ID NO: 7. In some embodiments, the frataxin expression construct comprises a frataxin exon 2-5 sequence encoding the amino acid sequence of SEQ ID NO: 15.

In some embodiments, the frataxin expression construct comprises a frataxin control region sequence, an exon 1 non-coding sequence, an exon 1 coding sequence, a frataxin intron 1 sequence, and a frataxin exon 2-5 sequence. In some embodiments, the frataxin expression construct comprises, from 5′ to 3′, a frataxin control region sequence, an exon 1 non-coding sequence, an exon 1 coding sequence, a frataxin intron 1 sequence, and a frataxin exon 2-5 sequence. In some embodiments, the frataxin expression construct comprises a frataxin control region sequence having the sequence of any one of SEQ ID NOs: 3, 16, and 17, an exon 1 non-coding sequence having the sequence of SEQ ID NO: 4, an exon 1 coding sequence having the sequence of SEQ ID NO: 5, a frataxin intron 1 sequence having the sequence of SEQ ID NO: 6, and a frataxin exon 2-5 sequence having the sequence of SEQ ID NO: 7. In some embodiments, the frataxin expression construct comprises, from 5′ to 3′, a frataxin control region sequence having the sequence of any one of SEQ ID NOs: 3, 16, and 17, an exon 1 non-coding sequence having the sequence of SEQ ID NO: 4, an exon 1 coding sequence having the sequence of SEQ ID NO: 5, a frataxin intron 1 sequence having the sequence of SEQ ID NO: 6, and a frataxin exon 2-5 sequence having the sequence of SEQ ID NO: 7.

In some embodiments the rAAV vector (e.g., nucleic acid or recombinant genome) further comprises additional sequences, such as inverted terminal repeat (ITR) sequences, an enhancer sequence, a 3′ untranslated region sequence, or a poly A sequence. In some embodiments, the left ITR sequence has the sequence of SEQ ID NO: 1 or SEQ ID NO: 11. In some embodiments, the right ITR sequence has the sequence of SEQ ID NO: 10. In some embodiments the rAAV vector comprises an enhancer sequence. In some embodiments, the enhancer sequence is an alpha-myosin heavy chain enhancer. In some embodiments, the enhancer sequence has the sequence of SEQ ID NO: 2. In some embodiments, the enhancer sequence is the desmin enhancer sequence. In some embodiments, the enhancer sequence has the sequence of SEQ ID NO: 13.

In some embodiments, the rAAV vector comprises the sequences as recited in Tables 1-5 shown below.

TABLE 1
Construct 1 (B908) (pTR2-P4-Fxn2a-Dual)

| | | SEQ | | SEQ |
|---|---|---|---|---|
| Elements | | ID | AA sequence | ID |
| (5′ -> 3′) | NT sequence | NO: | (as applicable) | NO: |
| ITR-L | TTGGCCACTCCCTCTCTGCGCG | 1 | | |
| | CTCGCTCGCTCACTGAGGCCGG | | | |
| | GCGACCAAAGGTCGCCCGACG | | | |
| | CCCGGGCTTTGCCCGGGCGGC | | | |
| | CTCAGTGAGCGAGCGAGCGCG | | | |
| | CAGAGAGGGAGTGGCCAACTC | | | |
| | CATCACTAGGGGTTCCT | | | |
| Alpha- | CCTTCAGATTAAAAATAACTA | 2 | | |
| myosin | AGGTAAGGGCCATGTGGGTAG | | | |
| heavy | GGGAGGTGGTGTGAGACGGTC | | | |
| chain | CTGTCTCTCCTCTATCTGCCCA | | | |
| enhancer | TCGGCCCTTTGGGGAGGAGGA | | | |
| | ATGTGCCCAAGGACTAAAAAA | | | |
| | AGGCCCTGGAGCCAGAGGGGC | | | |
| | GAGGGCAGCAGACCTTTCATG | | | |
| | GGCAAACCTCAGGGCTGCTGT | | | |
| | C | | | |
| Frataxin | ACAATCATAGCTCACTGCAGC | 3 | | |
| control | CTTGAGCTCCCAGGCTCAAGTG | | | |
| region | ATCCTCCCGCCTCAGCCTCCTG | | | |
| | AGTAGCTGAGATCACAGGCAT | | | |
| | GCACTACCACACTCGGCTCAC | | | |
| | ATTTGACATCCTCTAAAGCATA | | | |
| | TATAAAATGTGAAGAAAACTT | | | |
| | TCACAATTTGCATCCCTTTGTA | | | |
| | ATATGTAACAGAAATAAAATT | | | |
| | CTCTTTTAAAATCTATCAACAA | | | |
| | TAGGCAAGGCACGGTGGCTCA | | | |
| | CGCCTGTCGTCTCAGCACTTTG | | | |
| | TGAGGCCCAGGCGGGCAGATC | | | |
| | GTTTGAGCCTAGAAGTTCAAG | | | |
| | ACCACCCTGGCCAACATAGCG | | | |
| | AAACCCCCTTTCTACAAAAAAT | | | |
| | ACAAAAACTAGCTGGGTGTGG | | | |
| | TGGTGCACACCTGTAGTCCCAG | | | |
| | CTACTTGGAAGGCTGAAATGG | | | |
| | GAAGACTGCTTGAGCCCGGGA | | | |
| | GGGGGAAGTTGCAGTAAGCCA | | | |
| | GGACCACACCACTGCACTCCA | | | |
| | GCCTGGGCAACAGAGTGAGAC | | | |
| | TCTGTCTCAAACAAACAAATA | | | |
| | AATGAGGCGGGTGGATCACGA | | | |
| | GGTCAGTAGATCGAGACCATC | | | |
| | CTGGCTAACACGGTGAAACCC | | | |
| | GTCTCTACTAAAAAAAAAAAA | | | |
| | AAAATACAAAAAATTAGCCAG | | | |
| | GCATGGTGGCGGGCGCCTGTA | | | |
| | GTCCCAGTTACTCGGGAGGCT | | | |
| | GAGGCAGGAGAATGGCGTGAA | | | |
| | ACCGGGAGGCAGAGCTTGCAG | | | |
| | TGAGCCGAGATCGCACCACTG | | | |
| | CCCTCCAGCCTGGGCGACAGA | | | |
| | GCGAGACTCCGTCTCAATCAAT | | | |
| | CAATCAATCAATAAAATCTATT | | | |
| | AACAATATTTATTGTGCACTTA | | | |
| | ACAGGAACATGCCCTGTCCAA | | | |
| | AAAAACTTTACAGGGCTTAAC | | | |
| | TCATTTTATCCTTACCACAATC | | | |
| | CTATGAAGTAGGAACTTTTATA | | | |
| | AAACGCATTTTATAAACAAGG | | | |
| | CACAGAGAGGTTAATTAACTT | | | |
| | GCCCTCTGGTCACACAGCTAG | | | |
| | GAAGTGGGCAGAGTACAGATT | | | |
| | TACACAAGGCATCCGTCTCCTG | | | |
| | GCCCCACATACCCAACTGCTGT | | | |
| | AAACCCATACCGGCGGCCAAG | | | |
| | CAGCCTCAATTTGTGCATGCAC | | | |
| | CCACTTCCCAGCAAGACAGCA | | | |
| | GCTCCCAAGTTCCTCCTGTTTA | | | |
| | GAATTTTAGAAGCGGCGGGCC | | | |
| | ACCAGGCTGCTCTAG | | | |
| Exon1 | AGTCTCCCTTGGGTCAGGGGTC | 4 | | |
| non- | CTGGTTGCACTCCGTGCTTTGC | | | |
| coding | ACAAAGCAGGCTCTCCATTTTT | | | |
| | GTTAAATGCACGAATAGTGCT | | | |
| | AAGCTGGGAAGTTCTTCCTGA | | | |
| | GGTCTAACCTCTAGCTGCTCCC | | | |
| | CCACAGAAGAGTGCCTGCGGC | | | |
| | CAGTGGCCACCAGGGGTCGCC | | | |
| | GCAGCACCCAGCGCTGGAGGG | | | |
| | CGGAGCGGGCGGCAGACCCGG | | | |
| | AGCAGC | | | |
| Exon 1 | ATGTGGACCCTCGGCCGCAGA | 5 | MWTLGRRAVAGLLA | 14 |
| coding | GCTGTTGCTGGACTTCTTGCCT | | SPSPAQAQTLTRVPRP | |
| | CTCCATCTCCAGCTCAGGCCCA | | AELAPLCGRRGLRTDI | |
| | GACACTGACCAGAGTGCCTAG | | DATCTPRRA | |
| | ACCTGCTGAACTGGCCCCTCTG | | | |
| | TGTGGCAGAAGAGGCCTGAGA | | | |
| | ACCGACATCGACGCCACATGC | | | |
| | ACACCTAGAAGGGCC | | | |
| Frataxin | GTAAGTATCCGCGCCGGGAAC | 6 | | |
| Intron 1 | AGCCGCGGGCCGCACGCCGCG | | | |
| | GGCCGCACGCCGCACGCCTGC | | | |
| | GCAGGGAGGCGCCGCAATATA | | | |
| | TAAATTATGCATTAATGGGTTA | | | |
| | TAATTCACTGAAAAATAGTAA | | | |
| | CGTACTTCTTAACTTTGGCTTT | | | |
| | CAG | | | |
| Exon 2-5 | AGCAGCAATCAGCGGGGCCTG | 7 | SSNQRGLNQIWNVKK | 15 |
| | AATCAAATCTGGAACGTGAAG | | QSVYLMNLRKSGTLG | |
| | AAACAGAGCGTGTACCTGATG | | HPGSLDETTYERLAEE | |
| | AACCTGAGAAAGAGCGGCACC | | TLDSLAEFFEDLADKP | |
| | CTGGGACACCCTGGAAGCCTG | | YTFEDYDVSFGSGVLT | |
| | GATGAGACAACCTACGAGAGA | | VKLGGDLGTYVINKQ | |
| | CTGGCCGAGGAAACCCTGGAT | | TPNKQIWLSSPSSGPK | |
| | TCCCTGGCCGAGTTCTTCGAGG | | RYDWTGKNWVYSHD | |
| | ACCTGGCCGATAAGCCCTACA | | GVSLHELLAAELTKA | |
| | CCTTCGAGGATTACGACGTGTC | | LKTKLDLSSLAYSGK | |
| | CTTTGGCAGCGGCGTGCTGAC | | DA*** | |
| | AGTGAAACTCGGCGGAGATCT | | | |
| | GGGCACCTACGTGATCAACAA | | | |
| | GCAGACCCCTAACAAACAGAT | | | |
| | CTGGCTGAGCAGCCCTAGCAG | | | |
| | CGGCCCCAAGAGATATGATTG | | | |
| | GACCGGCAAGAACTGGGTGTA | | | |
| | CAGCCACGATGGCGTGTCCCT | | | |
| | GCACGAACTGCTGGCTGCCGA | | | |
| | ACTGACAAAGGCCCTGAAAAC | | | |
| | AAAGCTGGACCTGTCCAGCCT | | | |
| | GGCCTACTCTGGCAAGGACGC | | | |
| | CTGATGATGA | | | |
| 3′ | TCGAGTGCCCCAGCCCCGTTTT | 8 | | |
| Untrans- | AAGGACATTAAAAGCTATCAG | | | |
| lated | GCCAAGACCCCAGCTTCATTAT | | | |
| | GCAGCTGAGGTCTGTTTTTTGT | | | |
| | TGTTGTTGTTGTTTATTTTTTTT | | | |
| | ATTCCTGCTTTTGAGGACAGTT | | | |
| | GGGCTATGTGTCACAGCTCTGT | | | |
| | AGAAAGAATGTGTTGCCTCCT | | | |
| | ACCTTGCCCCCAAGTTCTGATT | | | |
| | TTTAATTTCTATGGAAGATTTT | | | |
| | TTGGATTGTCGGATTTCCTCCC | | | |
| | TCACATGATACCCCTTATCTTT | | | |
| | TATAATGTCTTATGCCTATACC | | | |
| | TGAATATAACAACCTTTAAAA | | | |
| | AAGCAAAATAATAAGAAGGAA | | | |
| | AAATTCCAGGAGGGAAAATGA | | | |
| | ATTGTCTTCACTCTTCATTCTTT | | | |
| | GAAGGATTTACTGCAAGAAGT | | | |
| | ACATGAAGAGCAGCTGGTCAA | | | |
| | CCTGCTCACTGTTCTATCTCCA | | | |
| | AATGAGACACATTAAAGGGTA | | | |
| | GCCTACAAATGTTTTCAGGCTT | | | |
| | CTTTCAAAGTGTAAGCACTTCT | | | |
| | GAGCTCTTTAGCATTGAAGTGT | | | |
| | CGAAAGCAACTCACACGGGAA | | | |
| | GATCATTTCTTATTTGTGCTCT | | | |
| | GTGACTGCCAAGGTGTGGCCT | | | |
| | GCACTGGGTTGTCCAGGGAGA | | | |
| | CATGCATCTAGTGCTGTTTCTC | | | |
| | CCACATATTCACATACGTGTCT | | | |
| | GTGTGTATATATATTTTTTCAA | | | |
| | TTTAAAGGTTAGTATGGAATCA | | | |
| | GCTGCTACAAGAATGCAAAAA | | | |
| | ATCTTCCAAAGACAAGAAAAG | | | |
| | AGGAAAAAAAGCCGTTTTCAT | | | |
| | GAGCTGAGTGATGTAGCGTAA | | | |
| | CAAACAAAATCATGGAGCTGA | | | |
| | GGAGGTGCCTTGTAAACATGA | | | |
| | AGGGGCAGATAAAGGAAGGA | | | |
| | GATACTCATGTTGATAAAGAG | | | |
| | AGCCCTGGTCCTAGACATAGTT | | | |
| | CAGCCACAAAGTAGTTGTCCCT | | | |
| | TTGTGGACAAGTTTCCCAAATT | | | |
| | CCCTGGACCTCTGCTTCCCCAT | | | |
| | CTGTTAAATGAGAGAATAGAG | | | |
| | TATGGTTGATTCCCAGCATTCA | | | |
| | GTGGTCCTGTCAAGCAACCTA | | | |
| | ACAGGCTAGTTCTAATTCCCTA | | | |
| | TTGGGTAGATGAGGGGATGAC | | | |
| | AAAGAACAGTTTTTAAGCTAT | | | |
| | ATAGGAAACATTGTTATTGGTG | | | |
| | TTGCCCTATCGTGATTTCAGTT | | | |
| | GAATTCATGTGAAAATAATAG | | | |
| | CCATCCTTGGCCTGGCGCGGTG | | | |
| | GCTCACACCTGTAATCCCAGCA | | | |
| | CTTTTGGAGGCCAAGGTGGGT | | | |
| | GGATCACCTGAGGTCAGGAGT | | | |
| | TCAAGACCAGCCTGGCCAACA | | | |
| | TGATGAAACCCCGTCTCTACTA | | | |
| | AAAATACAAAAAATTAGCCGG | | | |
| | GCATGATGGCAGGTGCCTGTA | | | |
| | ATCCCAGCTACTTGGGAGGCT | | | |
| | GAAGCGGAAGAATCGCTTGAA | | | |
| | CCCAGAGGTGGAGGTTGCAGT | | | |
| | GAGCCGAGATCGTGCCATTGC | | | |
| | ACTGTAACCTGGGTGACTGAG | | | |
| | CAAAACTCTGTCTCAAAATAAT | | | |
| | AATAACAATATAATAATAATA | | | |
| | ATAGCCATCCTTTATTGTACCC | | | |
| | TTACTGGGTTAATCGTATTATA | | | |
| | CCACATTACCTCATTTTAATTT | | | |
| | TTACTGACCTGCACTTTATACA | | | |
| | AAGCAACAAGCCTCCAGGACA | | | |
| | TTAAAATTCATGCAAAGTTATG | | | |
| | CTCATGTTATATTATTTTCTTAC | | | |
| | TTAAAGAAGGATTTATTAGTG | | | |
| | GCTGGGCATGGTGGCGTGCAC | | | |
| | CTGTAATCCCAGGTACTCAGG | | | |
| | AGGCTGAGACGGGAGAATTGC | | | |
| | TTGACCCCAGGCGGAGGAGGT | | | |
| | TACAGTGAGTCGAGATCGTAC | | | |
| | CTGAGCGACAGAGCGAGACTC | | | |
| | CGTCTCAAAAAAAAAAAAAAG | | | |
| | GAGGGTTTATTAATGAGAAGT | | | |
| | TTGG | | | |
| Poly A | CTGTGCCTTCTAGTTGCCAGCC | 9 | | |
| | ATCTGTTGTTTGCCCCTCCCCC | | | |
| | GTGCCTTCCTTGACCCTGGAAG | | | |
| | GTGCCACTCCCACTGTCCTTTC | | | |
| | CTAATAAAATGAGGAAATTGC | | | |
| | ATCGCATTGTCTGAGTAGGTGT | | | |
| | CATTCTATTCTGGGGGGTGGGG | | | |
| | TGGGGCAGGACAGCAAGGGGG | | | |
| | AGGATTGGGAAGACAATAGCA | | | |
| | GGCATGCTGGGGA | | | |
| ITR-R | AGGAACCCCTAGTGATGGAGT | 10 | | |
| | TGGCCACTCCCTCTCTGCGCGC | | | |
| | TCGCTCGCTCACTGAGGCCGG | | | |
| | GCGACCAAAGGTCGCCCGACG | | | |
| | CCCGGGCTTTGCCCGGGCGGC | | | |
| | CTCAGTGAGCGAGCGAGCGCG | | | |
| | CAGAGAGGGAGTGGCCAA | | | |

TABLE 2
Construct 2: (C063) (pdsTR2-Endo7-FXN2a-Frt-Hyg)

| | | SEQ | | SEQ |
|---|---|---|---|---|
| Elements | | ID | AA sequence (as | ID |
| (5′ -> 3′) | NT sequence | NO: | applicable) | NO: |
| ITR-delta | TTGGCCACTCCCTCTCTGCGCG | 11 | | |
| L | CTCGCTCGCTCACTGAGGCCGG | | | |
| | GCGACCAAAGGTCGCCCGACG | | | |
| | CCCGGGCTTTGCCCGGGCGGC | | | |
| | CTCAGTGAGCGAGCGAGCGCG | | | |
| | CAGAGAGGGAGTGGCCA | | | |
| Frataxin | ACAATCATAGCTCACTGCAGC | 3 | | |
| control | CTTGAGCTCCCAGGCTCAAGTG | | | |
| region | ATCCTCCCGCCTCAGCCTCCTG | | | |
| | AGTAGCTGAGATCACAGGCAT | | | |
| | GCACTACCACACTCGGCTCAC | | | |
| | ATTTGACATCCTCTAAAGCATA | | | |
| | TATAAAATGTGAAGAAAACTT | | | |
| | TCACAATTTGCATCCCTTTGTA | | | |
| | ATATGTAACAGAAATAAAATT | | | |
| | CTCTTTTAAAATCTATCAACAA | | | |
| | TAGGCAAGGCACGGTGGCTCA | | | |
| | CGCCTGTCGTCTCAGCACTTTG | | | |
| | TGAGGCCCAGGCGGGCAGATC | | | |
| | GTTTGAGCCTAGAAGTTCAAG | | | |
| | ACCACCCTGGCCAACATAGCG | | | |
| | AAACCCCCTTTCTACAAAAAAT | | | |
| | ACAAAAACTAGCTGGGTGTGG | | | |
| | TGGTGCACACCTGTAGTCCCAG | | | |
| | CTACTTGGAAGGCTGAAATGG | | | |
| | GAAGACTGCTTGAGCCCGGGA | | | |
| | GGGGGAAGTTGCAGTAAGCCA | | | |
| | GGACCACACCACTGCACTCCA | | | |
| | GCCTGGGCAACAGAGTGAGAC | | | |
| | TCTGTCTCAAACAAACAAATA | | | |
| | AATGAGGCGGGTGGATCACGA | | | |
| | GGTCAGTAGATCGAGACCATC | | | |
| | CTGGCTAACACGGTGAAACCC | | | |
| | GTCTCTACTAAAAAAAAAAAA | | | |
| | AAAATACAAAAAATTAGCCAG | | | |
| | GCATGGTGGCGGGCGCCTGTA | | | |
| | GTCCCAGTTACTCGGGAGGCT | | | |
| | GAGGCAGGAGAATGGCGTGAA | | | |
| | ACCGGGAGGCAGAGCTTGCAG | | | |
| | TGAGCCGAGATCGCACCACTG | | | |
| | CCCTCCAGCCTGGGCGACAGA | | | |
| | GCGAGACTCCGTCTCAATCAAT | | | |
| | CAATCAATCAATAAAATCTATT | | | |
| | AACAATATTTATTGTGCACTTA | | | |
| | ACAGGAACATGCCCTGTCCAA | | | |
| | AAAAACTTTACAGGGCTTAAC | | | |
| | TCATTTTATCCTTACCACAATC | | | |
| | CTATGAAGTAGGAACTTTTATA | | | |
| | AAACGCATTTTATAAACAAGG | | | |
| | CACAGAGAGGTTAATTAACTT | | | |
| | GCCCTCTGGTCACACAGCTAG | | | |
| | GAAGTGGGCAGAGTACAGATT | | | |
| | TACACAAGGCATCCGTCTCCTG | | | |
| | GCCCCACATACCCAACTGCTGT | | | |
| | AAACCCATACCGGCGGCCAAG | | | |
| | CAGCCTCAATTTGTGCATGCAC | | | |
| | CCACTTCCCAGCAAGACAGCA | | | |
| | GCTCCCAAGTTCCTCCTGTTTA | | | |
| | GAATTTTAGAAGCGGCGGGCC | | | |
| | ACCAGGCTGCTCTAG | | | |
| Exon1 | AGTCTCCCTTGGGTCAGGGGTC | 4 | | |
| non- | CTGGTTGCACTCCGTGCTTTGC | | | |
| coding | ACAAAGCAGGCTCTCCATTTTT | | | |
| | GTTAAATGCACGAATAGTGCT | | | |
| | AAGCTGGGAAGTTCTTCCTGA | | | |
| | GGTCTAACCTCTAGCTGCTCCC | | | |
| | CCACAGAAGAGTGCCTGCGGC | | | |
| | CAGTGGCCACCAGGGGTCGCC | | | |
| | GCAGCACCCAGCGCTGGAGGG | | | |
| | CGGAGCGGGCGGCAGACCCGG | | | |
| | AGCAGC | | | |
| Exon 1 | ATGTGGACCCTCGGCCGCAGA | 5 | MWTLGRRAVAGLLA | 14 |
| coding | GCTGTTGCTGGACTTCTTGCCT | | SPSPAQAQTLTRVPRP | |
| | CTCCATCTCCAGCTCAGGCCCA | | AELAPLCGRRGLRTDI | |
| | GACACTGACCAGAGTGCCTAG | | DATCTPRRA | |
| | ACCTGCTGAACTGGCCCCTCTG | | | |
| | TGTGGCAGAAGAGGCCTGAGA | | | |
| | ACCGACATCGACGCCACATGC | | | |
| | ACACCTAGAAGGGCC | | | |
| Frataxin | GTAAGTATCCGCGCCGGGAAC | 6 | | |
| Intron 1 | AGCCGCGGGCCGCACGCCGCG | | | |
| | GGCCGCACGCCGCACGCCTGC | | | |
| | GCAGGGAGGCGCCGCAATATA | | | |
| | TAAATTATGCATTAATGGGTTA | | | |
| | TAATTCACTGAAAAATAGTAA | | | |
| | CGTACTTCTTAACTTTGGCTTT | | | |
| | CAG | | | |
| Exon 2-5 | AGCAGCAATCAGCGGGGCCTG | 7 | SSNQRGLNQIWNVKK | 15 |
| | AATCAAATCTGGAACGTGAAG | | QSVYLMNLRKSGTLG | |
| | AAACAGAGCGTGTACCTGATG | | HPGSLDETTYERLAEE | |
| | AACCTGAGAAAGAGCGGCACC | | TLDSLAEFFEDLADKP | |
| | CTGGGACACCCTGGAAGCCTG | | YTFEDYDVSFGSGVLT | |
| | GATGAGACAACCTACGAGAGA | | VKLGGDLGTYVINKQ | |
| | CTGGCCGAGGAAACCCTGGAT | | TPNKQIWLSSPSSGPK | |
| | TCCCTGGCCGAGTTCTTCGAGG | | RYDWTGKNWVYSHD | |
| | ACCTGGCCGATAAGCCCTACA | | GVSLHELLAAELTKA | |
| | CCTTCGAGGATTACGACGTGTC | | LKTKLDLSSLAYSGK | |
| | CTTTGGCAGCGGCGTGCTGAC | | DA | |
| | AGTGAAACTCGGCGGAGATCT | | | |
| | GGGCACCTACGTGATCAACAA | | | |
| | GCAGACCCCTAACAAACAGAT | | | |
| | CTGGCTGAGCAGCCCTAGCAG | | | |
| | CGGCCCCAAGAGATATGATTG | | | |
| | GACCGGCAAGAACTGGGTGTA | | | |
| | CAGCCACGATGGCGTGTCCCT | | | |
| | GCACGAACTGCTGGCTGCCGA | | | |
| | ACTGACAAAGGCCCTGAAAAC | | | |
| | AAAGCTGGACCTGTCCAGCCT | | | |
| | GGCCTACTCTGGCAAGGACGC | | | |
| | CTGATGATGA | | | |
| Poly A | TAATAAAAGATCCTTATTTTCA | 12 | | |
| | TTGGATCTGTGTGTTGGTTTTT | | | |
| | TGTGTG | | | |
| ITR-R | AGGAACCCCTAGTGATGGAGT | 10 | | |
| | TGGCCACTCCCTCTCTGCGCGC | | | |
| | TCGCTCGCTCACTGAGGCCGG | | | |
| | GCGACCAAAGGTCGCCCGACG | | | |
| | CCCGGGCTTTGCCCGGGCGGC | | | |
| | CTCAGTGAGCGAGCGAGCGCG | | | |
| | CAGAGAGGGAGTGGCCAA | | | |

TABLE 3
Construct 3: (C096) (pdsTR2-DesEndo7-FXN2-Frt-Hyg (AAV9-DE7-hFXN))

| | | SEQ | | SEQ |
|---|---|---|---|---|
| Elements | | ID | AA sequence (as | ID |
| (5′ -> 3′) | NT sequence | NO: | applicable) | NO: |
| ITR-delta | TTGGCCACTCCCTCTCTGCG | 11 | | |
| L | CGCTCGCTCGCTCACTGAGG | | | |
| | CCGGGCGACCAAAGGTCGC | | | |
| | CCGACGCCCGGGCTTTGCCC | | | |
| | GGGCGGCCTCAGTGAGCGA | | | |
| | GCGAGCGCGCAGAGAGGGA | | | |
| | GTGGCCA | | | |
| Desmin | TCCCCCTGCCCCCACAGCTC | 13 | | |
| enhancer | CTCTCCTGTGCCTTGTTTCCC | | | |
| | AGCCATGCGTTCTCCTCTAT | | | |
| | AAATACCCGCTCTGGTATTT | | | |
| | GGGGTTGGCAGCTGTTGCTG | | | |
| | CCTGGGAGATGGTTGGGTTG | | | |
| | ACATGCGGCTCCTGACAAA | | | |
| | ACACAAACCCCTGGTGTGTG | | | |
| | TGGGCGTGGGTGGTGTGAGT | | | |
| | AGGGGGATGAATCAGGGAG | | | |
| | GGGGCGGGGGACCCAGGGG | | | |
| | GCAGGAGCCACACAAAGTC | | | |
| | TGTGCGGGGGTGGGAGCGC | | | |
| | ACATAGCAATTG | | | |
| Frataxin | TTGGAAGGCTGAAATGGGA | 17 | | |
| control | AGACTGCTTGAGCCCGGGA | | | |
| region | GGGGGAAGTTGCAGTAAGC | | | |
| | CAGGACCACACCACTGCACT | | | |
| | CCAGCCTGGGCAACAGAGT | | | |
| | GAGACTCTGTCTCAAACAAA | | | |
| | CAAATAAATGAGGCGGGTG | | | |
| | GATCACGAGGTCAGTAGAT | | | |
| | CGAGACCATCCTGGCTAACA | | | |
| | CGGTGAAACCCGTCTCTACT | | | |
| | AAAAAAAAAAAAAAAATAC | | | |
| | AAAAAATTAGCCAGGCATG | | | |
| | GTGGCGGGCGCCTGTAGTCC | | | |
| | CAGTTACTCGGGAGGCTGA | | | |
| | GGCAGGAGAATGGCGTGAA | | | |
| | ACCGGGAGGCAGAGCTTGC | | | |
| | AGTGAGCCGAGATCGCACC | | | |
| | ACTGCCCTCCAGCCTGGGCG | | | |
| | ACAGAGCGAGACTCCGTCTC | | | |
| | AATCAATCAATCAATCAATA | | | |
| | AAATCTATTAACAATATTTA | | | |
| | TTGTGCACTTAACAGGAACA | | | |
| | TGCCCTGTCCAAAAAAACTT | | | |
| | TACAGGGCTTAACTCATTTT | | | |
| | ATCCTTACCACAATCCTATG | | | |
| | AAGTAGGAACTTTTATAAAA | | | |
| | CGCATTTTATAAACAAGGCA | | | |
| | CAGAGAGGTTAATTTACTTG | | | |
| | CCCTCTGGTCACACAGCTAG | | | |
| | GAAGTGGGCAGAGTACAGA | | | |
| | TTTACACAAGGCATCCGTCT | | | |
| | CCTGGCCCCACATACCCAAC | | | |
| | TGCTGTAAACCCATACCGGC | | | |
| | GGCCAAGCAGCCTCAATTTG | | | |
| | TGCATGCACCCACTTCCCAG | | | |
| | CAAGACAGCAGCTCCCAAG | | | |
| | TTCCTCCTGTTTAGAATTTTA | | | |
| | GAAGCGGCGGGCCACCAGG | | | |
| | CTGCTCTAG | | | |
| Exon1 | AGTCTCCCTTGGGTCAGGGG | 4 | | |
| non- | TCCTGGTTGCACTCCGTGCT | | | |
| coding | TTGCACAAAGCAGGCTCTCC | | | |
| | ATTTTTGTTAAATGCACGAA | | | |
| | TAGTGCTAAGCTGGGAAGTT | | | |
| | CTTCCTGAGGTCTAACCTCT | | | |
| | AGCTGCTCCCCCACAGAAG | | | |
| | AGTGCCTGCGGCCAGTGGCC | | | |
| | ACCAGGGGTCGCCGCAGCA | | | |
| | CCCAGCGCTGGAGGGCGGA | | | |
| | GCGGGCGGCAGACCCGGAG | | | |
| | CAGC | | | |
| Exon 1 | ATGTGGACCCTCGGCCGCAG | 5 | MWTLGRRAVAGLL | 14 |
| coding | AGCTGTTGCTGGACTTCTTG | | ASPSPAQAQTLTRV | |
| | CCTCTCCATCTCCAGCTCAG | | PRPAELAPLCGRRG | |
| | GCCCAGACACTGACCAGAG | | LRTDIDATCTPRRA | |
| | TGCCTAGACCTGCTGAACTG | | | |
| | GCCCCTCTGTGTGGCAGAAG | | | |
| | AGGCCTGAGAACCGACATC | | | |
| | GACGCCACATGCACACCTA | | | |
| | GAAGGGCC | | | |
| Frataxin | GTAAGTATCCGCGCCGGGA | 6 | | |
| Intron 1 | ACAGCCGCGGGCCGCACGC | | | |
| | CGCGGGCCGCACGCCGCAC | | | |
| | GCCTGCGCAGGGAGGCGCC | | | |
| | GCAATATATAAATTATGCAT | | | |
| | TAATGGGTTATAATTCACTG | | | |
| | AAAAATAGTAACGTACTTCT | | | |
| | TAACTTTGGCTTTCAG | | | |
| Exon 2-5 | AGCAGCAATCAGCGGGGCC | 7 | SSNQRGLNQIWNVK | 15 |
| | TGAATCAAATCTGGAACGTG | | KQSVYLMNLRKSGT | |
| | AAGAAACAGAGCGTGTACC | | LGHPGSLDETTYER | |
| | TGATGAACCTGAGAAAGAG | | LAEETLDSLAEFFED | |
| | CGGCACCCTGGGACACCCTG | | LADKPYTFEDYDVS | |
| | GAAGCCTGGATGAGACAAC | | FGSGVLTVKLGGDL | |
| | CTACGAGAGACTGGCCGAG | | GTYVINKQTPNKQI | |
| | GAAACCCTGGATTCCCTGGC | | WLSSPSSGPKRYDW | |
| | CGAGTTCTTCGAGGACCTGG | | TGKNWVYSHDGVS | |
| | CCGATAAGCCCTACACCTTC | | LHELLAAELTKALK | |
| | GAGGATTACGACGTGTCCTT | | TKLDLSSLAYSGKD | |
| | TGGCAGCGGCGTGCTGACA | | A*** | |
| | GTGAAACTCGGCGGAGATC | | | |
| | TGGGCACCTACGTGATCAAC | | | |
| | AAGCAGACCCCTAACAAAC | | | |
| | AGATCTGGCTGAGCAGCCCT | | | |
| | AGCAGCGGCCCCAAGAGAT | | | |
| | ATGATTGGACCGGCAAGAA | | | |
| | CTGGGTGTACAGCCACGATG | | | |
| | GCGTGTCCCTGCACGAACTG | | | |
| | CTGGCTGCCGAACTGACAA | | | |
| | AGGCCCTGAAAACAAAGCT | | | |
| | GGACCTGTCCAGCCTGGCCT | | | |
| | ACTCTGGCAAGGACGCCTG | | | |
| | ATGATGA | | | |
| Poly A | TAATAAAAGATCCTTATTTT | 12 | | |
| | CATTGGATCTGTGTGTTGGT | | | |
| | TTTTTGTGTG | | | |
| ITR-R | AGGAACCCCTAGTGATGGA | 10 | | |
| | GTTGGCCACTCCCTCTCTGC | | | |
| | GCGCTCGCTCGCTCACTGAG | | | |
| | GCCGGGCGACCAAAGGTCG | | | |
| | CCCGACGCCCGGGCTTTGCC | | | |
| | CGGGCGGCCTCAGTGAGCG | | | |
| | AGCGAGCGCGCAGAGAGGG | | | |
| | AGTGGCCAA | | | |

TABLE 4
Construct 4: (C118) (pTR2-P6-FXN2-3′UTR)

| Elements | | SEQ ID | AA sequence (as | SEQ ID |
|---|---|---|---|---|
| (5′ -> 3′) | NT sequence | NO: | applicable) | NO: |
| ITR-L | TTGGCCACTCCCTCTCTGCGCG | 1 | | |
| | CTCGCTCGCTCACTGAGGCCGG | | | |
| | GCGACCAAAGGTCGCCCGACG | | | |
| | CCCGGGCTTTGCCCGGGCGGC | | | |
| | CTCAGTGAGCGAGCGAGCGCG | | | |
| | CAGAGAGGGAGTGGCCAACTC | | | |
| | CATCACTAGGGGTTCCT | | | |
| Frataxin | GATATCGGTACCATTTAGCATC | 16 | | |
| control | CTAAGTATGTAAACATGACTCT | | | |
| region | CTTCACGATTCACAAAGTGGCT | | | |
| | TTGGAAGAACTTTAGTACCTTC | | | |
| | CCATCTTCTCTGCCATGGAAAG | | | |
| | TGTACACAACTGACATTTTCTT | | | |
| | TTTTTTAAGACAGTATCTTGCT | | | |
| | ATGATGGCCGGGCTGGAATGC | | | |
| | TGTGGCTATTCACAGGCACAAT | | | |
| | CATAGCTCACTGCAGCCTTGAG | | | |
| | CTCCCAGGCTCAAGTGATCCTC | | | |
| | CCGCCTCAGCCTCCTGAGTAGC | | | |
| | TGAGATCACAGGCATGCACTA | | | |
| | CCACACTCGGCTCACATTTGAC | | | |
| | ATCCTCTAAAGCATATATAAA | | | |
| | ATGTGAAGAAAACTTTCACAA | | | |
| | TTTGCATCCCTTTGTAATATGT | | | |
| | AACAGAAATAAAATTCTCTTTT | | | |
| | AAAATCTATCAACAATAGGCA | | | |
| | AGGCACGGTGGCTCACGCCTG | | | |
| | TCGTCTCAGCACTTTGTGAGGC | | | |
| | CCAGGCGGGCAGATCGTTTGA | | | |
| | GCCTAGAAGTTCAAGACCACC | | | |
| | CTGGCCAACATAGCGAAACCC | | | |
| | CCTTTCTACAAAAAATACAAA | | | |
| | AACTAGCTGGGTGTGGTGGTG | | | |
| | CACACCTGTAGTCCCAGCTACT | | | |
| | TGGAAGGCTGAAATGGGAAGA | | | |
| | CTGCTTGAGCCCGGGAGGGGG | | | |
| | AAGTTGCAGTAAGCCAGGACC | | | |
| | ACACCACTGCACTCCAGCCTG | | | |
| | GGCAACAGAGTGAGACTCTGT | | | |
| | CTCAAACAAACAAATAAATGA | | | |
| | GGCGGGTGGATCACGAGGTCA | | | |
| | GTAGATCGAGACCATCCTGGC | | | |
| | TAACACGGTGAAACCCGTCTCT | | | |
| | ACTAAAAAAAAAAAAAAAATA | | | |
| | CAAAAAATTAGCCAGGCATGG | | | |
| | TGGCGGGCGCCTGTAGTCCCA | | | |
| | GTTACTCGGGAGGCTGAGGCA | | | |
| | GGAGAATGGCGTGAAACCGGG | | | |
| | AGGCAGAGCTTGCAGTGAGCC | | | |
| | GAGATCGCACCACTGCCCTCC | | | |
| | AGCCTGGGCGACAGAGCGAGA | | | |
| | CTCCGTCTCAATCAATCAATCA | | | |
| | ATCAATAAAATCTATTAACAAT | | | |
| | ATTTATTGTGCACTTAACAGGA | | | |
| | ACATGCCCTGTCCAAAAAAAC | | | |
| | TTTACAGGGCTTAACTCATTTT | | | |
| | ATCCTTACCACAATCCTATGAA | | | |
| | GTAGGAACTTTTATAAAACGC | | | |
| | ATTTTATAAACAAGGCACAGA | | | |
| | GAGGTTAATTAACTTGCCCTCT | | | |
| | GGTCACACAGCTAGGAAGTGG | | | |
| | GCAGAGTACAGATTTACACAA | | | |
| | GGCATCCGTCTCCTGGCCCCAC | | | |
| | ATACCCAACTGCTGTAAACCC | | | |
| | ATACCGGCGGCCAAGCAGCCT | | | |
| | CAATTTGTGCATGCACCCACTT | | | |
| | CCCAGCAAGACAGCAGCTCCC | | | |
| | AAGTTCCTCCTGTTTAGAATTT | | | |
| | TAGAAGCGGCGGGCCACCAGG | | | |
| | CTGCTCTAG | | | |
| Exon1 | AGTCTCCCTTGGGTCAGGGGTC | 4 | | |
| non- | CTGGTTGCACTCCGTGCTTTGC | | | |
| coding | ACAAAGCAGGCTCTCCATTTTT | | | |
| | GTTAAATGCACGAATAGTGCT | | | |
| | AAGCTGGGAAGTTCTTCCTGA | | | |
| | GGTCTAACCTCTAGCTGCTCCC | | | |
| | CCACAGAAGAGTGCCTGCGGC | | | |
| | CAGTGGCCACCAGGGGTCGCC | | | |
| | GCAGCACCCAGCGCTGGAGGG | | | |
| | CGGAGCGGGCGGCAGACCCGG | | | |
| | AGCAGC | | | |
| Exon 1 | ATGTGGACCCTCGGCCGCAGA | 5 | MWTLGRRAVAGLLA | 14 |
| coding | GCTGTTGCTGGACTTCTTGCCT | | SPSPAQAQTLTRVPRP | |
| | CTCCATCTCCAGCTCAGGCCCA | | AELAPLCGRRGLRTDI | |
| | GACACTGACCAGAGTGCCTAG | | DATCTPRRA | |
| | ACCTGCTGAACTGGCCCCTCTG | | | |
| | TGTGGCAGAAGAGGCCTGAGA | | | |
| | ACCGACATCGACGCCACATGC | | | |
| | ACACCTAGAAGGGCC | | | |
| Frataxin | GTAAGTATCCGCGCCGGGAAC | 6 | | |
| Intron 1 | AGCCGCGGGCCGCACGCCGCG | | | |
| | GGCCGCACGCCGCACGCCTGC | | | |
| | GCAGGGAGGCGCCGCAATATA | | | |
| | TAAATTATGCATTAATGGGTTA | | | |
| | TAATTCACTGAAAAATAGTAA | | | |
| | CGTACTTCTTAACTTTGGCTTT | | | |
| | CAG | | | |
| Exon 2-5 | AGCAGCAATCAGCGGGGCCTG | 7 | SSNQRGLNQIWNVKK | 15 |
| | AATCAAATCTGGAACGTGAAG | | QSVYLMNLRKSGTLG | |
| | AAACAGAGCGTGTACCTGATG | | HPGSLDETTYERLAEE | |
| | AACCTGAGAAAGAGCGGCACC | | TLDSLAEFFEDLADKP | |
| | CTGGGACACCCTGGAAGCCTG | | YTFEDYDVSFGSGVLT | |
| | GATGAGACAACCTACGAGAGA | | VKLGGDLGTYVINKQ | |
| | CTGGCCGAGGAAACCCTGGAT | | TPNKQIWLSSPSSGPK | |
| | TCCCTGGCCGAGTTCTTCGAGG | | RYDWTGKNWVYSHD | |
| | ACCTGGCCGATAAGCCCTACA | | GVSLHELLAAELTKA | |
| | CCTTCGAGGATTACGACGTGTC | | LKTKLDLSSLAYSGK | |
| | CTTTGGCAGCGGCGTGCTGAC | | DA*** | |
| | AGTGAAACTCGGCGGAGATCT | | | |
| | GGGCACCTACGTGATCAACAA | | | |
| | GCAGACCCCTAACAAACAGAT | | | |
| | CTGGCTGAGCAGCCCTAGCAG | | | |
| | CGGCCCCAAGAGATATGATTG | | | |
| | GACCGGCAAGAACTGGGTGTA | | | |
| | CAGCCACGATGGCGTGTCCCT | | | |
| | GCACGAACTGCTGGCTGCCGA | | | |
| | ACTGACAAAGGCCCTGAAAAC | | | |
| | AAAGCTGGACCTGTCCAGCCT | | | |
| | GGCCTACTCTGGCAAGGACGC | | | |
| | CTGATGATGA | | | |
| 3′ | TCGAGTGCCCCAGCCCCGTTTT | 8 | | |
| Untrans- | AAGGACATTAAAAGCTATCAG | | | |
| lated | GCCAAGACCCCAGCTTCATTAT | | | |
| | GCAGCTGAGGTCTGTTTTTTGT | | | |
| | TGTTGTTGTTGTTTATTTTTTTT | | | |
| | ATTCCTGCTTTTGAGGACAGTT | | | |
| | GGGCTATGTGTCACAGCTCTGT | | | |
| | AGAAAGAATGTGTTGCCTCCT | | | |
| | ACCTTGCCCCCAAGTTCTGATT | | | |
| | TTTAATTTCTATGGAAGATTTT | | | |
| | TTGGATTGTCGGATTTCCTCCC | | | |
| | TCACATGATACCCCTTATCTTT | | | |
| | TATAATGTCTTATGCCTATACC | | | |
| | TGAATATAACAACCTTTAAAA | | | |
| | AAGCAAAATAATAAGAAGGAA | | | |
| | AAATTCCAGGAGGGAAAATGA | | | |
| | ATTGTCTTCACTCTTCATTCTTT | | | |
| | GAAGGATTTACTGCAAGAAGT | | | |
| | ACATGAAGAGCAGCTGGTCAA | | | |
| | CCTGCTCACTGTTCTATCTCCA | | | |
| | AATGAGACACATTAAAGGGTA | | | |
| | GCCTACAAATGTTTTCAGGCTT | | | |
| | CTTTCAAAGTGTAAGCACTTCT | | | |
| | GAGCTCTTTAGCATTGAAGTGT | | | |
| | CGAAAGCAACTCACACGGGAA | | | |
| | GATCATTTCTTATTTGTGCTCT | | | |
| | GTGACTGCCAAGGTGTGGCCT | | | |
| | GCACTGGGTTGTCCAGGGAGA | | | |
| | CATGCATCTAGTGCTGTTTCTC | | | |
| | CCACATATTCACATACGTGTCT | | | |
| | GTGTGTATATATATTTTTTCAA | | | |
| | TTTAAAGGTTAGTATGGAATCA | | | |
| | GCTGCTACAAGAATGCAAAAA | | | |
| | ATCTTCCAAAGACAAGAAAAG | | | |
| | AGGAAAAAAAGCCGTTTTCAT | | | |
| | GAGCTGAGTGATGTAGCGTAA | | | |
| | CAAACAAAATCATGGAGCTGA | | | |
| | GGAGGTGCCTTGTAAACATGA | | | |
| | AGGGGCAGATAAAGGAAGGA | | | |
| | GATACTCATGTTGATAAAGAG | | | |
| | AGCCCTGGTCCTAGACATAGTT | | | |
| | CAGCCACAAAGTAGTTGTCCCT | | | |
| | TTGTGGACAAGTTTCCCAAATT | | | |
| | CCCTGGACCTCTGCTTCCCCAT | | | |
| | CTGTTAAATGAGAGAATAGAG | | | |
| | TATGGTTGATTCCCAGCATTCA | | | |
| | GTGGTCCTGTCAAGCAACCTA | | | |
| | ACAGGCTAGTTCTAATTCCCTA | | | |
| | TTGGGTAGATGAGGGGATGAC | | | |
| | AAAGAACAGTTTTTAAGCTAT | | | |
| | ATAGGAAACATTGTTATTGGTG | | | |
| | TTGCCCTATCGTGATTTCAGTT | | | |
| | GAATTCATGTGAAAATAATAG | | | |
| | CCATCCTTGGCCTGGCGCGGTG | | | |
| | GCTCACACCTGTAATCCCAGCA | | | |
| | CTTTTGGAGGCCAAGGTGGGT | | | |
| | GGATCACCTGAGGTCAGGAGT | | | |
| | TCAAGACCAGCCTGGCCAACA | | | |
| | TGATGAAACCCCGTCTCTACTA | | | |
| | AAAATACAAAAAATTAGCCGG | | | |
| | GCATGATGGCAGGTGCCTGTA | | | |
| | ATCCCAGCTACTTGGGAGGCT | | | |
| | GAAGCGGAAGAATCGCTTGAA | | | |
| | CCCAGAGGTGGAGGTTGCAGT | | | |
| | GAGCCGAGATCGTGCCATTGC | | | |
| | ACTGTAACCTGGGTGACTGAG | | | |
| | CAAAACTCTGTCTCAAAATAAT | | | |
| | AATAACAATATAATAATAATA | | | |
| | ATAGCCATCCTTTATTGTACCC | | | |
| | TTACTGGGTTAATCGTATTATA | | | |
| | CCACATTACCTCATTTTAATTT | | | |
| | TTACTGACCTGCACTTTATACA | | | |
| | AAGCAACAAGCCTCCAGGACA | | | |
| | TTAAAATTCATGCAAAGTTATG | | | |
| | CTCATGTTATATTATTTTCTTAC | | | |
| | TTAAAGAAGGATTTATTAGTG | | | |
| | GCTGGGCATGGTGGCGTGCAC | | | |
| | CTGTAATCCCAGGTACTCAGG | | | |
| | AGGCTGAGACGGGAGAATTGC | | | |
| | TTGACCCCAGGCGGAGGAGGT | | | |
| | TACAGTGAGTCGAGATCGTAC | | | |
| | CTGAGCGACAGAGCGAGACTC | | | |
| | CGTCTCAAAAAAAAAAAAAAG | | | |
| | GAGGGTTTATTAATGAGAAGT | | | |
| | TTGG | | | |
| Poly A | CTGTGCCTTCTAGTTGCCAGCC | 9 | | |
| | ATCTGTTGTTTGCCCCTCCCCC | | | |
| | GTGCCTTCCTTGACCCTGGAAG | | | |
| | GTGCCACTCCCACTGTCCTTTC | | | |
| | CTAATAAAATGAGGAAATTGC | | | |
| | ATCGCATTGTCTGAGTAGGTGT | | | |
| | CATTCTATTCTGGGGGGTGGGG | | | |
| | TGGGGCAGGACAGCAAGGGGG | | | |
| | AGGATTGGGAAGACAATAGCA | | | |
| | GGCATGCTGGGGA | | | |
| ITR-R | AGGAACCCCTAGTGATGGAGT | 10 | | |
| | TGGCCACTCCCTCTCTGCGCGC | | | |
| | TCGCTCGCTCACTGAGGCCGG | | | |
| | GCGACCAAAGGTCGCCCGACG | | | |
| | CCCGGGCTTTGCCCGGGCGGC | | | |
| | CTCAGTGAGCGAGCGAGCGCG | | | |
| | CAGAGAGGGAGTGGCCAA | | | |

TABLE 5
Construct 5: (C151) (pdsTR2-DesEndo7-FXN2a-KanV2)

| | | SEQ | | |
|---|---|---|---|---|
| Elements | | ID | AA sequence (as | SEQ ID |
| (5′ -> 3′) | NT sequence | NO: | applicable) | NO: |
| ITR-delta | TTGGCCACTCCCTCTCTGCGCG | 11 | | |
| L | CTCGCTCGCTCACTGAGGCCGG | | | |
| | GCGACCAAAGGTCGCCCGACG | | | |
| | CCCGGGCTTTGCCCGGGCGGC | | | |
| | CTCAGTGAGCGAGCGAGCGCG | | | |
| | CAGAGAGGGAGTGGCCA | | | |
| Desmin | TCCCCCTGCCCCCACAGCTCCT | 13 | | |
| Enhancer | CTCCTGTGCCTTGTTTCCCAGC | | | |
| | CATGCGTTCTCCTCTATAAATA | | | |
| | CCCGCTCTGGTATTTGGGGTTG | | | |
| | GCAGCTGTTGCTGCCTGGGAG | | | |
| | ATGGTTGGGTTGACATGCGGCT | | | |
| | CCTGACAAAACACAAACCCCT | | | |
| | GGTGTGTGTGGGCGTGGGTGG | | | |
| | TGTGAGTAGGGGGATGAATCA | | | |
| | GGGAGGGGGCGGGGGACCCAG | | | |
| | GGGGCAGGAGCCACACAAAGT | | | |
| | CTGTGCGGGGGTGGGAGCGCA | | | |
| | CATAGCAATTG | | | |
| Frataxin | TTGGAAGGCTGAAATGGGAAG | 17 | | |
| control | ACTGCTTGAGCCCGGGAGGGG | | | |
| region | GAAGTTGCAGTAAGCCAGGAC | | | |
| | CACACCACTGCACTCCAGCCTG | | | |
| | GGCAACAGAGTGAGACTCTGT | | | |
| | CTCAAACAAACAAATAAATGA | | | |
| | GGCGGGTGGATCACGAGGTCA | | | |
| | GTAGATCGAGACCATCCTGGC | | | |
| | TAACACGGTGAAACCCGTCTCT | | | |
| | ACTAAAAAAAAAAAAAAAATA | | | |
| | CAAAAAATTAGCCAGGCATGG | | | |
| | TGGCGGGCGCCTGTAGTCCCA | | | |
| | GTTACTCGGGAGGCTGAGGCA | | | |
| | GGAGAATGGCGTGAAACCGGG | | | |
| | AGGCAGAGCTTGCAGTGAGCC | | | |
| | GAGATCGCACCACTGCCCTCC | | | |
| | AGCCTGGGCGACAGAGCGAGA | | | |
| | CTCCGTCTCAATCAATCAATCA | | | |
| | ATCAATAAAATCTATTAACAAT | | | |
| | ATTTATTGTGCACTTAACAGGA | | | |
| | ACATGCCCTGTCCAAAAAAAC | | | |
| | TTTACAGGGCTTAACTCATTTT | | | |
| | ATCCTTACCACAATCCTATGAA | | | |
| | GTAGGAACTTTTATAAAACGC | | | |
| | ATTTTATAAACAAGGCACAGA | | | |
| | GAGGTTAATTTACTTGCCCTCT | | | |
| | GGTCACACAGCTAGGAAGTGG | | | |
| | GCAGAGTACAGATTTACACAA | | | |
| | GGCATCCGTCTCCTGGCCCCAC | | | |
| | ATACCCAACTGCTGTAAACCC | | | |
| | ATACCGGCGGCCAAGCAGCCT | | | |
| | CAATTTGTGCATGCACCCACTT | | | |
| | CCCAGCAAGACAGCAGCTCCC | | | |
| | AAGTTCCTCCTGTTTAGAATTT | | | |
| | TAGAAGCGGCGGGCCACCAGG | | | |
| | CTGCTCTAG | | | |
| Exon1 | AGTCTCCCTTGGGTCAGGGGTC | 4 | | |
| non- | CTGGTTGCACTCCGTGCTTTGC | | | |
| coding | ACAAAGCAGGCTCTCCATTTTT | | | |
| | GTTAAATGCACGAATAGTGCT | | | |
| | AAGCTGGGAAGTTCTTCCTGA | | | |
| | GGTCTAACCTCTAGCTGCTCCC | | | |
| | CCACAGAAGAGTGCCTGCGGC | | | |
| | CAGTGGCCACCAGGGGTCGCC | | | |
| | GCAGCACCCAGCGCTGGAGGG | | | |
| | CGGAGCGGGCGGCAGACCCGG | | | |
| | AGCAGC | | | |
| Exon 1 | ATGTGGACCCTCGGCCGCAGA | 5 | MWTLGRRAVAGLLA | 14 |
| coding | GCTGTTGCTGGACTTCTTGCCT | | SPSPAQAQTLTRVPR | |
| | CTCCATCTCCAGCTCAGGCCCA | | PAELAPLCGRRGLRT | |
| | GACACTGACCAGAGTGCCTAG | | DIDATCTPRRA | |
| | ACCTGCTGAACTGGCCCCTCTG | | | |
| | TGTGGCAGAAGAGGCCTGAGA | | | |
| | ACCGACATCGACGCCACATGC | | | |
| | ACACCTAGAAGGGCC | | | |
| Frataxin | GTAAGTATCCGCGCCGGGAAC | 6 | | |
| Intron 1 | AGCCGCGGGCCGCACGCCGCG | | | |
| | GGCCGCACGCCGCACGCCTGC | | | |
| | GCAGGGAGGCGCCGCAATATA | | | |
| | TAAATTATGCATTAATGGGTTA | | | |
| | TAATTCACTGAAAAATAGTAA | | | |
| | CGTACTTCTTAACTTTGGCTTT | | | |
| | CAG | | | |
| Exon 2-5 | AGCAGCAATCAGCGGGGCCTG | 7 | SSNQRGLNQIWNVK | 15 |
| | AATCAAATCTGGAACGTGAAG | | KQSVYLMNLRKSGT | |
| | AAACAGAGCGTGTACCTGATG | | LGHPGSLDETTYERL | |
| | AACCTGAGAAAGAGCGGCACC | | AEETLDSLAEFFEDL | |
| | CTGGGACACCCTGGAAGCCTG | | ADKPYTFEDYDVSFG | |
| | GATGAGACAACCTACGAGAGA | | SGVLTVKLGGDLGT | |
| | CTGGCCGAGGAAACCCTGGAT | | YVINKQTPNKQIWLS | |
| | TCCCTGGCCGAGTTCTTCGAGG | | SPSSGPKRYDWTGKN | |
| | ACCTGGCCGATAAGCCCTACA | | WVYSHDGVSLHELL | |
| | CCTTCGAGGATTACGACGTGTC | | AAELTKALKTKLDLS | |
| | CTTTGGCAGCGGCGTGCTGAC | | SLAYSGKDA*** | |
| | AGTGAAACTCGGCGGAGATCT | | | |
| | GGGCACCTACGTGATCAACAA | | | |
| | GCAGACCCCTAACAAACAGAT | | | |
| | CTGGCTGAGCAGCCCTAGCAG | | | |
| | CGGCCCCAAGAGATATGATTG | | | |
| | GACCGGCAAGAACTGGGTGTA | | | |
| | CAGCCACGATGGCGTGTCCCT | | | |
| | GCACGAACTGCTGGCTGCCGA | | | |
| | ACTGACAAAGGCCCTGAAAAC | | | |
| | AAAGCTGGACCTGTCCAGCCT | | | |
| | GGCCTACTCTGGCAAGGACGC | | | |
| | CTGATGATGA | | | |
| Poly A | TAATAAAAGATCCTTATTTTCA | 12 | | |
| | TTGGATCTGTGTGTTGGTTTTT | | | |
| | TGTGTG | | | |
| ITR-R | AGGAACCCCTAGTGATGGAGT | 10 | | |
| | TGGCCACTCCCTCTCTGCGCGC | | | |
| | TCGCTCGCTCACTGAGGCCGG | | | |
| | GCGACCAAAGGTCGCCCGACG | | | |
| | CCCGGGCTTTGCCCGGGCGGC | | | |
| | CTCAGTGAGCGAGCGAGCGCG | | | |
| | CAGAGAGGGAGTGGCCAA | | | |

rAAV Particles

In some aspects, the present disclosure contemplates rAAV vectors encapsidated in rAAV particles. The rAAV particle may be of any AAV serotype (e.g., 1, 2, 3, 4, 5, 6, 7, 8, 9, 10, 11, 12, or 13), including any derivative (including non-naturally occurring variants of a serotype) or pseudotype. Non-limiting examples of derivatives and pseudotypes include AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV218, AAV-HSC15/17, AAVM41, AAV9.45, AAV6 (Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol Ther. 2012 April; 20 (4): 699-708. doi: 10.1038/mt.2011.287. Epub 2012 Jan. 24. The AAV vector toolkit: poised at the clinical crossroads. Asokan Al, Schaffer D V, Samulski R J.). In some embodiments, the rAAV particle is a pseudotyped rAAV particle, which comprises (a) a nucleic acid vector comprising ITRs from one serotype (e.g., AAV2) and (b) a capsid comprised of capsid proteins derived from another serotype (e.g., AAV1, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Producing rAAV Particles

Methods of producing rAAV particles and nucleic acid vectors are also known in the art and commercially available (see, e.g., Zolotukhin et al., Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, the nucleic acid vector (e.g., as a plasmid) may be combined with one or more helper plasmids, e.g., that contain a rep gene (e.g., encoding Rep78, Rep68, Rep52 and Rep40) and a cap gene (encoding VP1, VP2, and VP3), and transfected into a producer cell line such that the rAAV particle can be packaged and subsequently purified.

In some embodiments, the packaging is performed in a helper cell or producer cell, such as a mammalian cell or an insect cell. Exemplary mammalian cells include, but are not limited to, HEK293 cells, COS cells, HeLa cells, BHK cells, or CHO cells (see, e.g., ATCC® CRL-1573™, ATCC® CRL-1651™, ATCC® CRL-1650™, ATCC® CCL-2, ATCC® CCL-10™, or ATCC® CCL-61™). Exemplary insect cells include, but are not limited to Sf9 cells (see, e.g., ATCC® CRL-1711™). The helper cell may comprise rep and/or cap genes that encode the Rep protein and/or Cap proteins for use in a method described herein. In some embodiments, the packaging is performed in vitro.

In some embodiments, the one or more helper plasmids includes a first helper plasmid comprising a rep gene and a cap gene and a second helper plasmid comprising other genes that assist in AAV production, such as a E1a gene, a E1b gene, a E4 gene, a E2a gene, and a VA gene. In some embodiments, the rep gene is a rep gene derived from AAV2 and the cap gene is derived from AAV5. Helper plasmids, and methods of making such plasmids, are known in the art and commercially available (see, e.g., pDF6, pRep, pDM, pDG, pDP1rs, pDP2rs, pDP3rs, pDP4rs, pDP5rs, pDP6rs, pDG (R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adenoassociated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. Alternatively, in another example, Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

The disclosure also contemplates host cells that comprise at least one of the disclosed rAAV particles, expression constructs, or nucleic acid vectors. Such host cells include mammalian host cells, with human host cells being preferred, and may be either isolated, in cell or tissue culture. In the case of genetically modified animal models (e.g., a mouse), the transformed host cells may be comprised within the body of a non-human animal itself.

### Compositions

Aspects of the disclosure relate to compositions comprising rAAV particles or nucleic acids described herein. In some embodiments, rAAV particles described herein are added to a composition, e.g., a pharmaceutical composition.

In some embodiments, the composition comprises a pharmaceutically acceptable carrier. The term “carrier” refers to a diluent, adjuvant, excipient, or vehicle with which the rAAV particle is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases). Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of rAAV particles to human subjects. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Methods for making such compositions are well known and can be found in, for example, Remington: The Science and Practice of Pharmacy, 22nd edition, Pharmaceutical Press, 2012.

Typically, such compositions may contain at least about 0.1% of the therapeutic agent (e.g., rAAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., rAAV particle) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, a composition described herein may be administered to a subject in need thereof, such as a subject having or suspected of having Friedreich's ataxia. In some embodiments, a method described herein may comprise administering a composition comprising rAAV particles as described herein to a subject in need thereof. In some embodiments, the subject is a human subject. In some embodiments, the subject has or is suspected of having a disease that may be treated with gene therapy, such as Friedreich's ataxia.

### Methods

Aspects of the disclosure relate to treatment of diseases resulting from aberrant frataxin protein levels (e.g., Friedreich's ataxia). In some embodiments, the method comprises administering a therapeutically effective amount of an rAAV particle or a composition as described herein to a subject having or suspected of having Friedreich's ataxia.

In some embodiments, to “treat” a disease as the term is used herein, means to reduce the frequency or severity of at least one sign or symptom of a disease or disorder experienced by a subject. In some embodiments, to “treat” a disease means delaying the onset of the disease (e.g., delaying the onset of one or more symptoms of the disease), preventing the onset of the disease (e.g., preventing the onset of one or more symptoms of the disease), or slowing the progression of the disease (e.g., lessening the severity of one or more symptoms of the disease relative to an untreated subject at a similar stage of the disease).

The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host organ, tissue, or cell. A therapeutically acceptable amount may be an amount that is capable of treating a disease, e.g., Friedreich's ataxia. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

Aspects of the disclosure relate to methods of increasing the level of frataxin in a cell or a subject. In some embodiments, the cell is a mammalian cell. In some embodiments, the cell is a human cell. In some embodiments, the cell is a cultured cell. In some embodiments, the cell is a cell in a subject. In some embodiments, the subject is a mammal. In some embodiments, the subject is a mouse. In some embodiments, the subject is a non-human primate. In some embodiments, the subject is a human. In some embodiments, the cell or subject has or is suspected of having a disorder related to low levels of frataxin (e.g., Friedreich's ataxia).

In some embodiments, the rAAV vectors described herein produce a desired level of frataxin expression. In some embodiments, a high level of frataxin expression is beneficial. In some embodiments, a moderate level of frataxin expression (e.g., a level of frataxin expression similar to the level of frataxin in a healthy individual without FRDA (“wildtype level”)) is beneficial. In some embodiments, a high level of frataxin expression in certain tissues, such as heart tissue, is detrimental to a subject. In some embodiments, the rAAV vectors described herein produce a level of frataxin expression that is closer to a wildtype level of frataxin than an alternative rAAV vector. In some embodiments, an alternative rAAV vector does not comprise one or more elements of the presently described rAAV vectors. In some embodiments, an alternative rAAV vector does not comprise a frataxin control region (such as any one of SEQ ID NOs: 3, 16, and 17) or native frataxin promoter. In some embodiments, an alternative rAAV vector does not comprise an exon 1 noncoding region (such as SEQ ID NO: 4). In some embodiments, an alternative rAAV vector does not comprise a frataxin intron (such as SEQ ID NO: 6)

The rAAV particle or nucleic acid vector may be delivered in the form of a composition, such as a composition comprising the active ingredient, such as a rAAV particle described herein, and a pharmaceutically acceptable carrier as described herein. The rAAV particles or nucleic acid vectors may be prepared in a variety of compositions, and may also be formulated in appropriate pharmaceutical vehicles for administration to human or animal subjects.

In some embodiments, rAAV particles are administered to a subject in a composition comprising rAAV particles at a concentration on the order ranging from 10⁶ to 10¹⁴ particles/ml or 10³ to 10¹⁵ particles/ml, or any values therebetween for either range, such as for example, at a concentration of about 10⁶, 10⁷, 10⁸, 10⁹, 10¹⁰, 10¹¹, 10¹², 10¹³, or 10¹⁴ particles/ml. In one embodiment, a composition comprising rAAV particles at a concentration higher than 10¹³ particles/ml id administered. In some embodiments, the number of rAAV particles administered to a subject may be on the order ranging from 10⁶ to 10¹⁴ vector genomes (vgs)/ml or 10³ to 10¹⁵ vgs/ml, or any values therebetween for either range, such as for example, about 10⁶, 10⁷, 10⁸, 10⁹, 10¹⁰, 10¹¹, 10¹², 10¹³, or 10¹⁴ vgs/ml. In one embodiment, rAAV particles of higher than 10¹³ vgs/ml are be administered. The rAAV particles can be administered as a single dose, or divided into two or more administrations as may be required to achieve therapy of the particular disease or disorder being treated. In some embodiments, 0.0001 ml to 10 mls are delivered to a subject. In some embodiments, the number of rAAV particles administered to a subject may be on the order ranging from 10⁶-10¹⁴ vg/kg, or any values therebetween, such as for example, about 10⁶, 10⁷, 10⁸, 10⁹, 10¹⁰, 10¹¹, 10¹², 10¹³, or 10¹⁴ vgs/kg. In some embodiments, the number of rAAV particles administered to a subject may be on the order ranging from 10¹²-10¹⁴ vgs/kg.

If desired, rAAV particles may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles may thus be delivered along with various other agents as required in the particular instance. Such compositions may be purified from host cells or other biological sources, or alternatively may be chemically synthesized.

In certain circumstances it will be desirable to deliver the rAAV particles in suitably formulated pharmaceutical compositions disclosed herein either subcutaneously, intraocularly, intravitreally, subretinally, parenterally, intravenously (IV), intracerebro-ventricularly, intramuscularly, intrathecally (IT), intracisternally, orally, intraperitoneally, by oral or nasal inhalation, or by direct injection to one or more cells, tissues, or organs by direct injection. In some embodiments, the administration is a route suitable for systemic delivery, such as by intravenous injection. In some embodiments, “administering” or “administration” means providing a material to a subject in a manner that is pharmacologically useful.

To address the systemic manifestations of FRDA, approaches to transfer AAV vectors globally that target both the neurological and cardiac impairment may be needed. Although IV dosing is an advantageous route to transduce the heart, it does not have high translational feasibility for CNS disorders because of the high dose requirement, high distribution to peripheral tissues and reduced efficiency for CNS transduction (Schuster, D. et al., Front Neuronat., 8:42, 2014). However, intrathecal (IT) dosing of AAV9 (e.g., via lumbar cistern or cisterna magna) is a viable, clinically relevant option for global CNS gene delivery (Gray, S. et al., Gene Ther, 20 (4): 450-9, 2013; Federici, T. et al., Gene Ther, 19 (8): 852, 2012; Snyder, B. et al., Hum Gene Ther, 22 (9): 1129, 2011). A method of treating subjects with FRDA using a combination of different routes of administration (e.g., IV and IT), by transduction of cardiac muscle, pancreas (e.g., pancreatic islet cells), and CNS (e.g., dorsal root ganglia, and the cerebellum) is contemplated herein. Thus, in some embodiments, rAAV particles or compositions comprising rAAV particles that comprise (or package) FXN transgene are administered via intravenous (IV) injection. In some embodiments, rAAV particles or compositions comprising rAAV particles that comprise (or package) FXN transgene are administered via intrathecal (IT) injection. In some embodiments, rAAV particles or compositions comprising rAAV particles that comprise (or package) FXN transgene are administered via intracisternal injection, so as to deliver the rAAV particles within a cistern of the brain. In some embodiments, rAAV particles or compositions comprising rAAV particles that package FXN transgene are administered via both an intravenous injection and an intrathecal injection, or via an intravenous injection and an intracisternal injection.

In some embodiments, more than two (e.g., three, or four) of the above-described routes of administration are utilized.

In some embodiments, the ratio of rAAV particles administered to the subject via intravenous injection to rAAV particles administered to the subject via intrathecal injection is in the range of 10:1 to 1:10 (e.g., 10:1, 8:1, 5:1, 4:1, 2:1, 1:1, 1:2, 1:5, 1:8, 1:10), or 50:1 to 1:50 (e.g., 50:1, 40:1, 25:1, 30:1, 10:1, 1:1, 1:10, 1:30, 1:25, 1:40, 1:50), or 100:1 to 1:100 (e.g., 100:1, 80:1, 50:1, 10:1, 1:1, 1:10, 1:50, 1:80, 1:100), or 1000:1 to 1:1000 (e.g., 1000:1, 800:1, 500:1, 100:1, 1:1, 1:100, 1:500, 1:800, 1:1000). In some embodiments, the ratio of rAAV particles administered to the subject via intravenous injection to rAAV particles administered to the subject via intracisternal injection is in the range of 10:1 to 1:10 (e.g., 10:1, 8:1, 5:1, 4:1, 2:1, 1:1, 1:2, 1:5, 1:8, 1:10), or 50:1 to 1:50 (e.g., 50:1, 40:1, 25:1, 30:1, 10:1, 1:1, 1:10, 1:30, 1:25, 1:40, 1:50), or 100:1 to 1:100 (e.g., 100:1, 80:1, 50:1, 10:1, 1:1, 1:10, 1:50, 1:80, 1:100), or 1000:1 to 1:1000 (e.g., 1000:1, 800:1, 500:1, 100:1, 1:1, 1:100, 1:500, 1:800, 1:1000). In some embodiments, the ratio of rAAV particles administered to the subject via intravenous injection to rAAV particles administered to the subject via intrathecal injection is 1:10. In some embodiments, the ratio of rAAV particles administered to the subject via intravenous injection to rAAV particles administered to the subject via intracisternal injection is 1:10. In some embodiments, compositions administered via intravenous, intrathecal, and/or intracisternal injection may have the same number of particles or viral genomes per unit volume. However, in some embodiments, compositions having different titers may be used for different routes of administration. Also, different compositions having different components in addition to the viral particles may be used for different routes of administration.

The amount of rAAV particle or nucleic acid vector compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the rAAV particle compositions, either over a relatively short, or a relatively prolonged period of time, as may be determined by the medical practitioner overseeing the administration of such compositions.

In some embodiments, administration of the rAAV particle or nucleic acid vector compositions further comprises administration of an immunosuppressive regimen. In some embodiments, an immunosuppressive regimen comprises administration of one or more immunosuppressive compounds to a subject. In some embodiments, one or more immunosuppressive compounds are administered before administration of the rAAV particle or nucleic acid vector composition. In some embodiments, one or more immunosuppressive compounds are administered during administration of the rAAV particle or nucleic acid vector composition. In some embodiments, one or more immunosuppressive compounds are administered after administration of the rAAV particle or nucleic acid vector composition. In some embodiments, the one or more immunosuppressive compounds comprise an anti-CD20 agent, such as an antibody. In some embodiments, the one or more immunosuppressive compounds comprise Sirolimus. In some embodiments, the one or more immunosuppressive compounds comprise Belimumab. In some embodiments, an immunosuppressive regiment comprises administration of two or more of an anti-CD20 agent, Sirolimus, and Belimumab. In some embodiments, an immunosuppressive regiment comprises administration of an anti-CD20 agent and Sirolimus. In some embodiments, an immunosuppressive regiment comprises administration of Sirolimus and Belimumab.

In some embodiments, when more than one route of administration is utilized, the administration of rAAV comprising FXN transgene via the two or more routes is performed simultaneously, or within 10 min of each other. In some embodiments, when more than one route of administration is utilized, the administration of rAAV comprising FXN transgene via the two or more routes is staggered, so that administration via the second route is performed 10 min, 20 min, 30 min 1 h, 2 h, 3 h, 4 h, 5 h, 6 h, 8 h, 12 h, 18 h, or 24 h after the administration via the first route. In some embodiments, when more than one route of administration is utilized, the administration of rAAV comprising FXN transgene via the two or more routes is altered, so that administration via the second route replaces administration via the first route on a routine basis (e.g., for once a day schedule, administration via first route on day 1, administration via second route on day 2, administration via first route on day 3, administration via second route on day 4, and so on).

In some embodiments, the number of rAAV particles administered to a subject may be on the order ranging from 10⁶ to 10¹⁴ particles/mL or 10³ to 10¹³ particles/mL, or any values therebetween for either range, such as for example, about 10⁶, 10⁷, 10⁸, 10⁹, 10¹⁰, 10¹¹, 10¹², 10¹³, or 10¹⁴ particles/mL. In some embodiments, rAAV particle compositions of lower than 10⁷ particles/mL, for example lower than 10³ particles/mL, are administered. In some embodiments, rAAV particle compositions of higher than 10¹³ particles/mL, for example higher than 10¹⁵ particles/mL, are administered. In some embodiments, the number of rAAV particles administered to a subject may be on the order ranging from 10⁶ to 10¹⁴ vector genomes (vgs)/mL or 10³ to 10¹⁵ vgs/mL, or any values there between for either range, such as for example, about 10⁶, 10⁷, 10⁸, 10⁹, 10¹⁰, 10¹¹, 10¹², 10¹³, or 10¹⁴ vgs/mL. In some embodiments, rAAV particle compositions of lower than 10⁷ vgs/mL, for example lower than 10³ vgs/mL, are be administered. In some embodiments, rAAV particle compositions of higher than 10¹³ vgs/mL, for example higher than 10¹⁵ vgs/mL, are administered. In some embodiments, 0.0001 mL to 10 mLs are delivered to a subject (e.g., via one or more routes of administration as described in this application).

IV and IT injections are routine non-surgical procedures that are often done in an outpatient setting with minimal risk (Mattar, C. et al., FASEB J, 29 (9): 3876, 2015; Gray, S. et al., Gene Ther, 20 (4): 450-9, 2013; Federici, T. et al., Gene Ther, 19 (8): 852, 2012; Snyder, B. et al., Hum Gene Ther, 22 (9): 1129, 2011).

The pharmaceutical forms of the rAAV particle compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. In some embodiments, the form is sterile. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subretinal, subcutaneous and intraperitoneal administration. In this connection, a sterile aqueous medium that can be employed will be known to those of skill in the art, in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles in the required amount in the appropriate solvent with several of the other ingredients enumerated above, as required, followed by filtered sterilization or another sterilization technique. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The composition may include rAAV particles, either alone, or in combination with one or more additional active ingredients, which may be obtained from natural or recombinant sources or chemically synthesized.

Toxicity and efficacy of the compositions utilized in methods of the disclosure can be determined by standard pharmaceutical procedures, using either cells in culture or experimental animals to determine the LD50 (the dose lethal to 50% of the population). The dose ratio between toxicity and efficacy the therapeutic index and it can be expressed as the ratio LD50/ED50. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED50 with little or no toxicity. The dosage may vary within this range depending upon the dosage form employed and the route of administration utilized.

### Subjects

Aspects of the disclosure relate to methods for use with a subject, such as human or non-human primate subjects. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some embodiments, the subject has or is suspected of having a disease that may be treated with gene therapy. In some embodiments, the subject has or is suspected of having Friedreich's ataxia. Friedreich's ataxia (FRDA) is a rare inherited disease that causes degeneration of the spinal cord and peripheral nervous system. Subjects with FRDA generally have an expanded number of GAA repeats in the FXN gene. A subject generally must have both copies of the FXN with expanded repeats to develop FRDA, although about 2 percent of subjects have one copy of the FXN gene with expanded repeats and another different type of mutation in the other copy of the FXN gene. Generally, if a subject has more than 66 to more than 1,000 GAA repeats in both copies of the FXN gene, they will develop FRDA. Symptoms of FRDA include gait ataxia, loss of sensation in the extremities, loss of tendon reflexes, scoliosis, dysarthria, hearing loss, vision loss, chest pain, shortness of breath, and heart palpitations. Subjects with FRDA may also develop carbohydrate intolerance or diabetes. Subjects with fewer than 300 repeats may develop symptoms later in life than those with additional repeats. Subject having FRDA can be identified by the skilled practitioner using methods known in the art or described herein, e.g., using genetic testing, electromyogram (EMG), nerve conduction studies, electrocardiogram (ECG), echocardiogram, blood tests for elevated glucose and vitamin E, magnetic resonance imaging (MRI) or computed tomography (CT) scans, and combinations thereof.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1. Materials and Methods

### Construct Design.

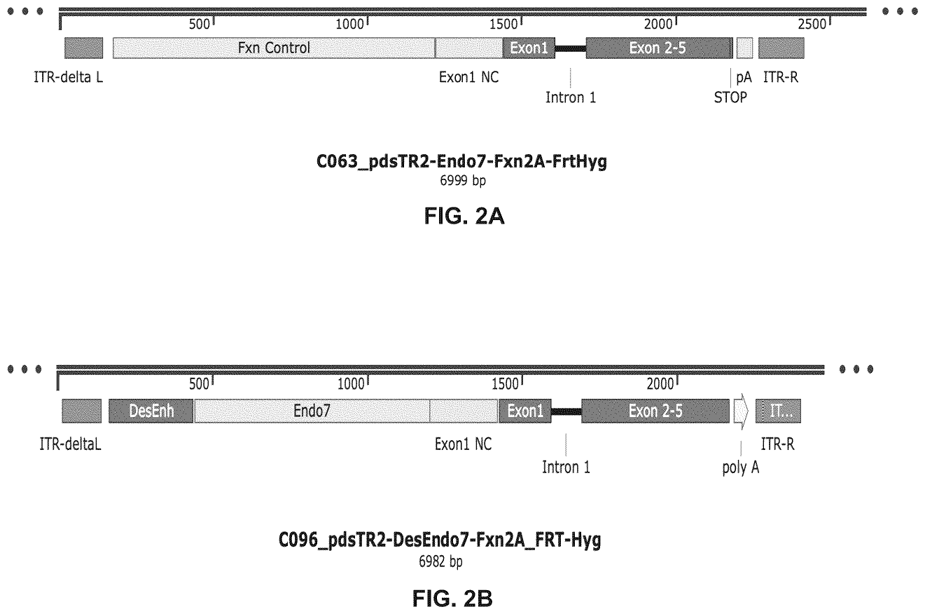

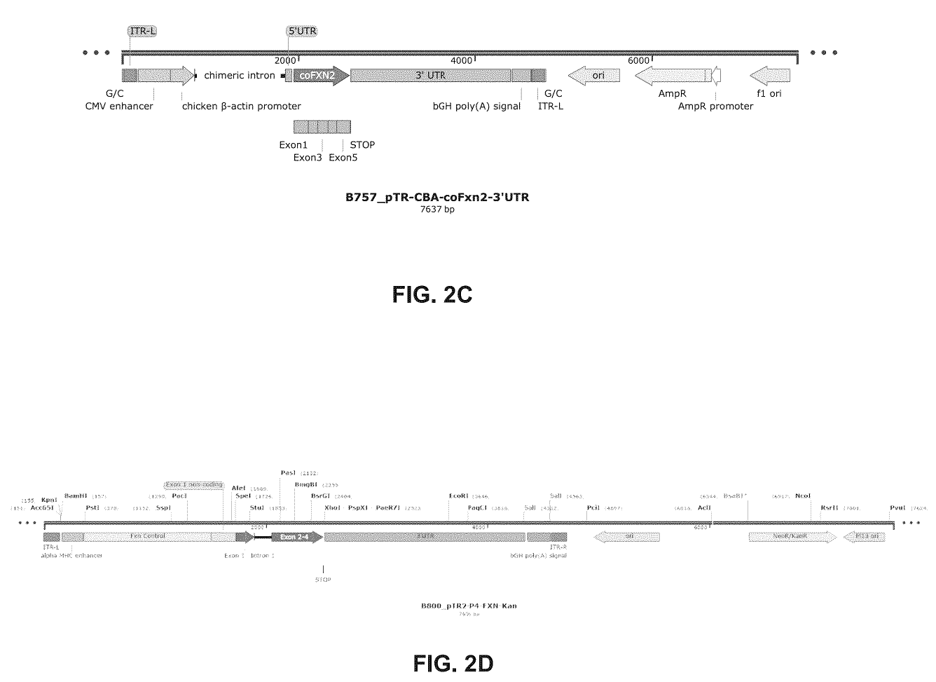

Expression cassettes which code for human frataxin utilizing regulatory sequences of the native frataxin in a configuration that leads to optimal frataxin expression and avoiding frataxin overexpression. The constructs were engineered to comprise the elements as provided in the Tables 1-5. Schematic representations of the constructs are provided in FIGS. 2A-2D.

### AAV Production.

Recombinant AAV (rAAV) particles comprising each of the constructs are made by suspension transfection of Expi293T cells. AAV vectors were isolated using a capture column followed by an anion exchange column and purified using a cesium chloride gradient to a titer of 2-5E+13 vg/ml.

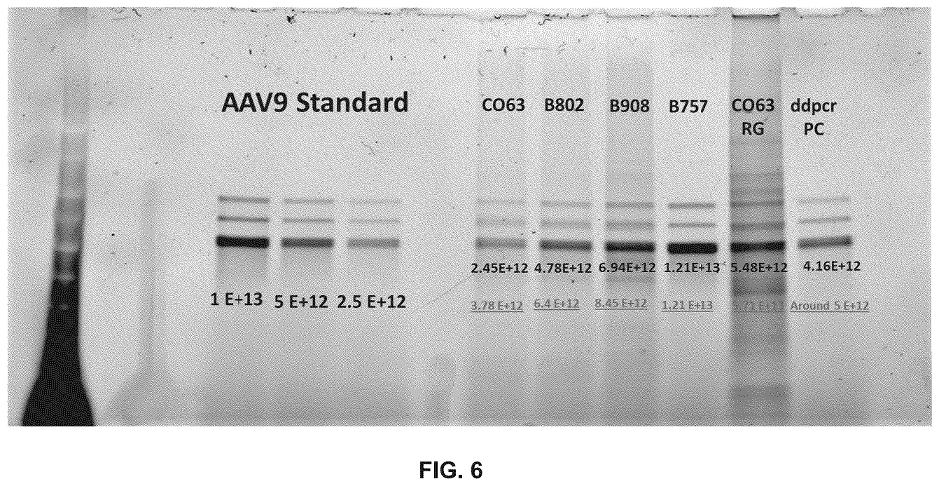

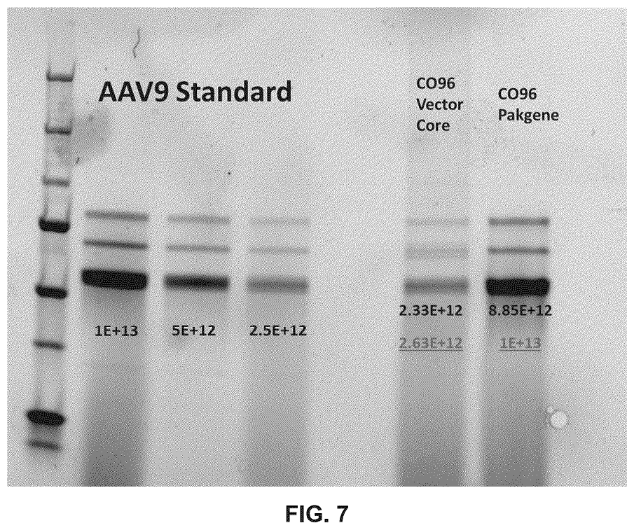

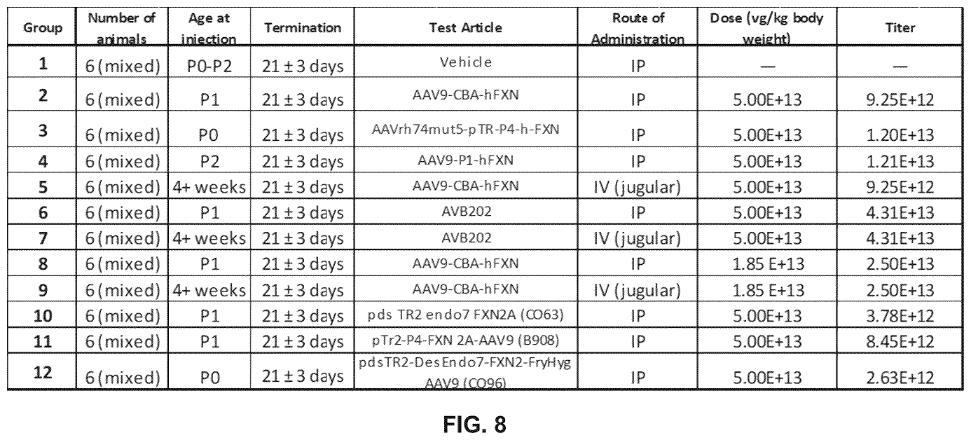

AAV vector packaging and titer was assessed relative to an AAV9 standard using a stain free gel as shown in FIGS. 6-7.

### Frataxin Expression In Vitro

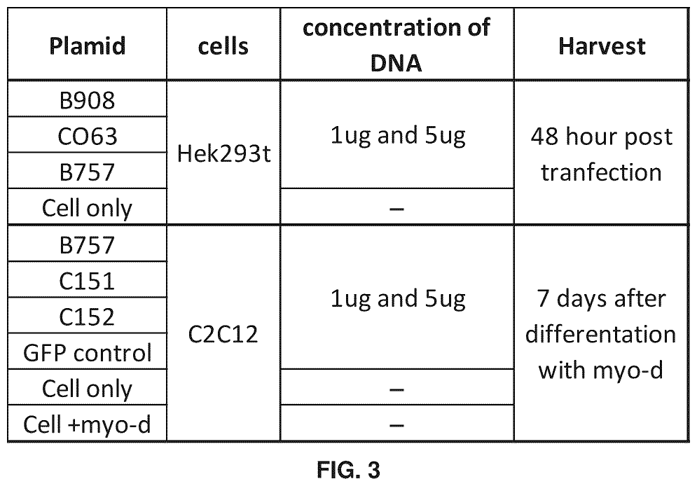

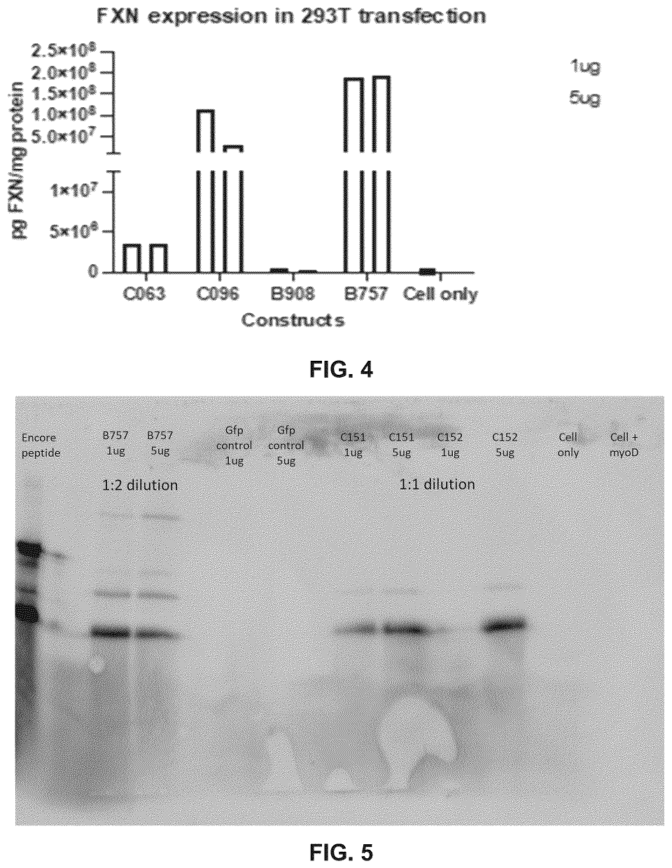

Expression of frataxin by non-limiting examples of expression vectors described herein was measured in 293T cells. Vectors described in FIG. 3 were transfected into HEK 293T cells and C2C12 cells using the concentration of DNA shown in FIG. 3 and standard methods. Results are shown in FIG. 4 (293T cells) and FIG. 5 (C2C12 cells).

### Frataxin Expression In Vivo

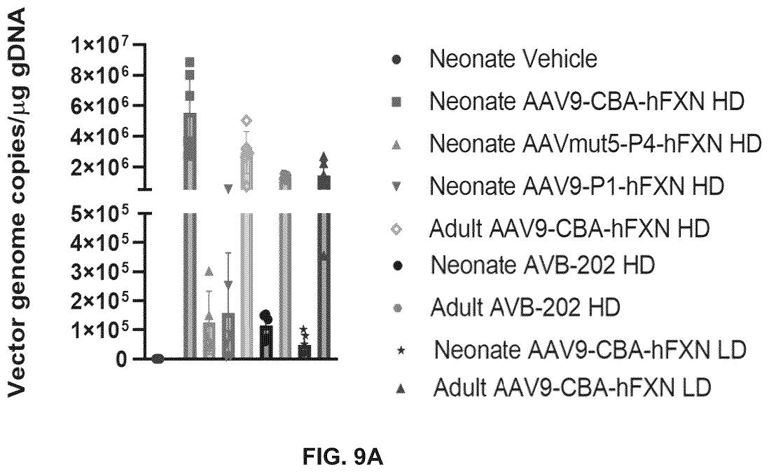

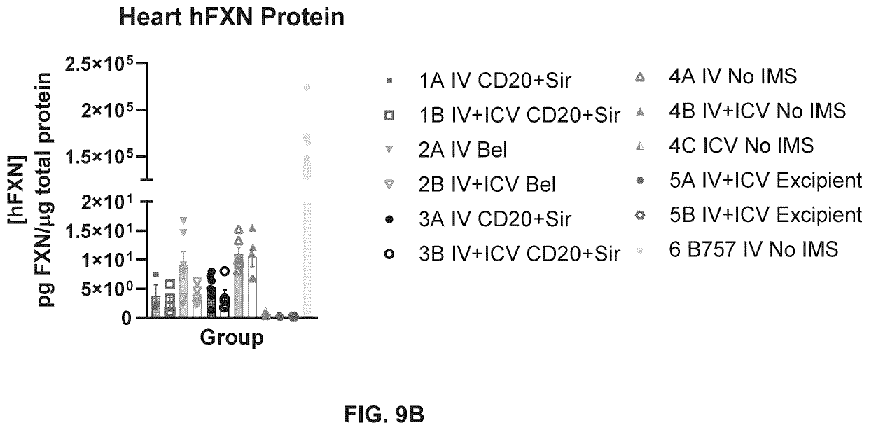

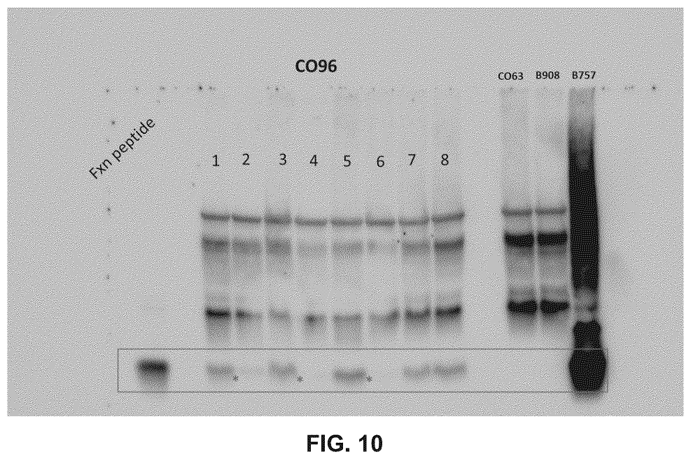

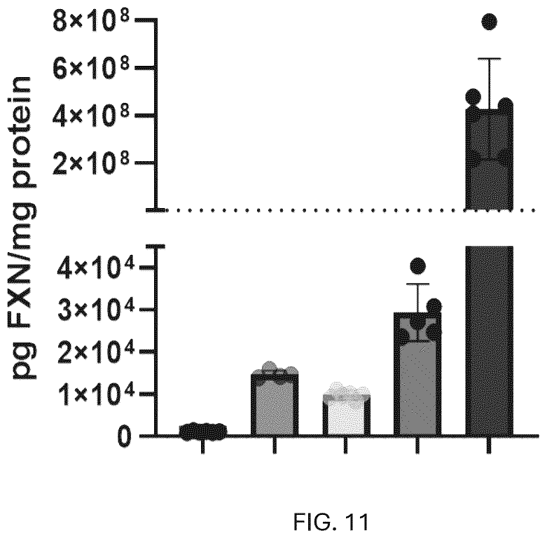

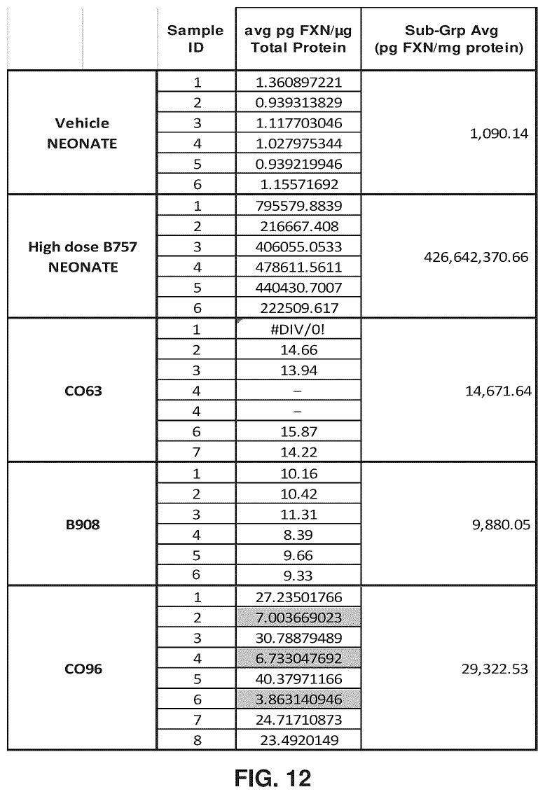

Expression of frataxin by non-limiting examples of expression vectors described herein was measured in a neonatal mouse model. Mice were dosed as shown in 8. Levels of frataxin expressed in the heart of neonatal mice were measured. Results are shown in FIGS. 9-12.

### Immunosuppression Study

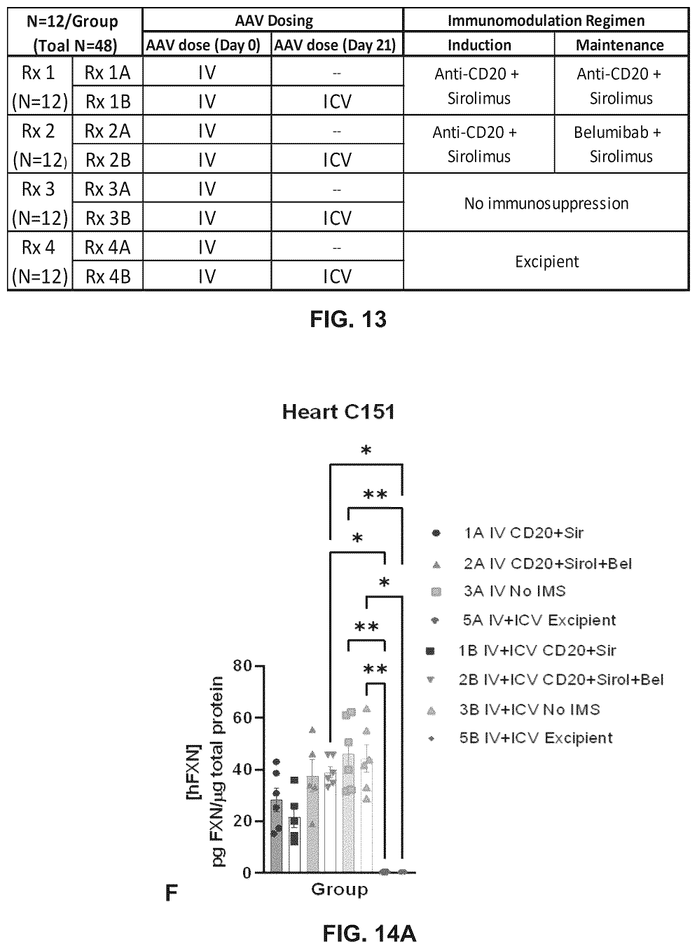

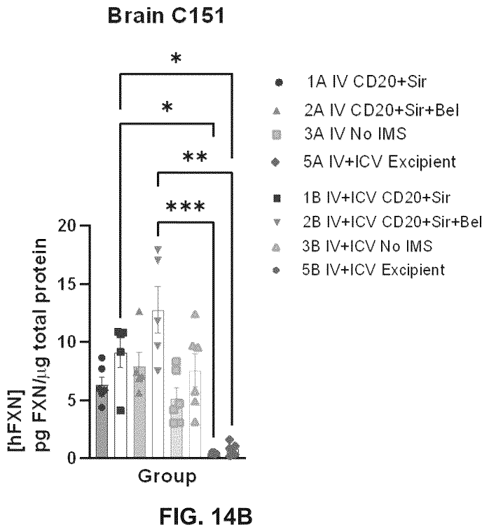

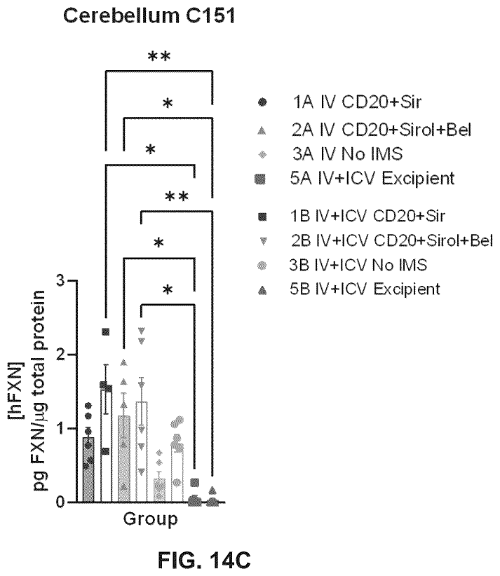

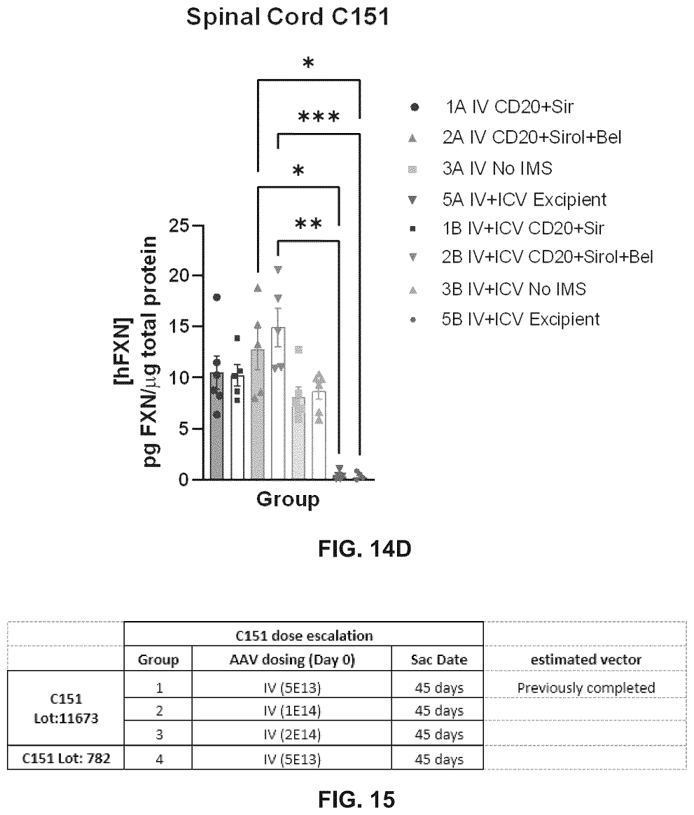

The effect of administering an immunosuppressive regimen on expression of frataxin and redosing using vectors as described herein was tested. Mice were treated as described in FIG. 13. Results are shown in FIGS. 14A-D.

### Dose Escalation

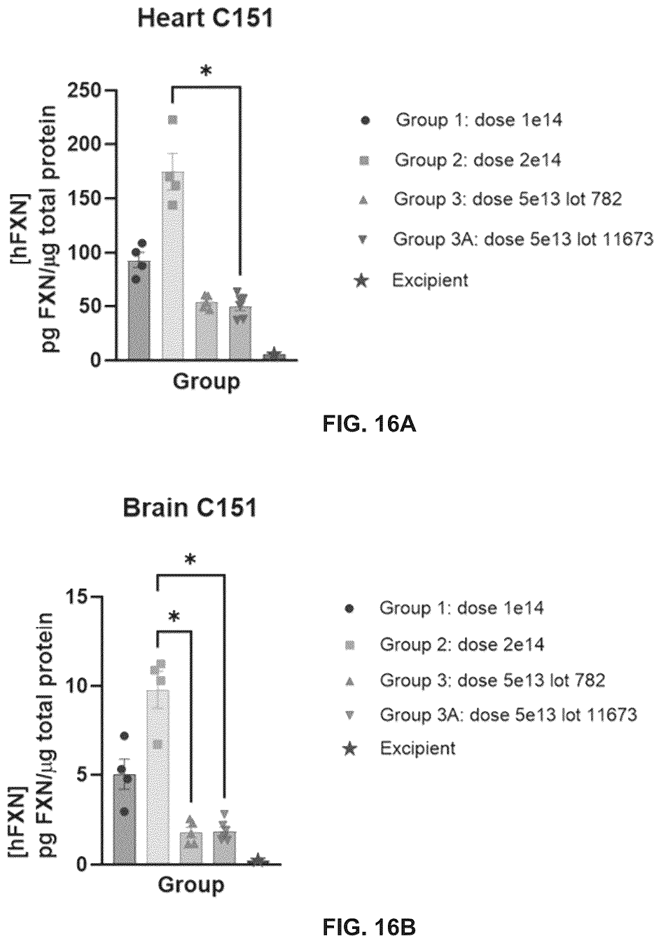

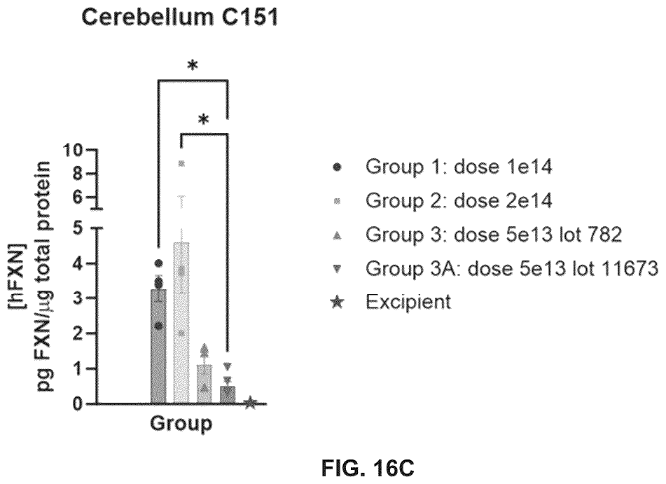

Escalating doses of frataxin-encoding rAAV vectors as described herein were tested in mice. Mice were treated as described in FIG. 15. Results are shown in FIGS. 16A-16B.

### Non-Human Primate Study

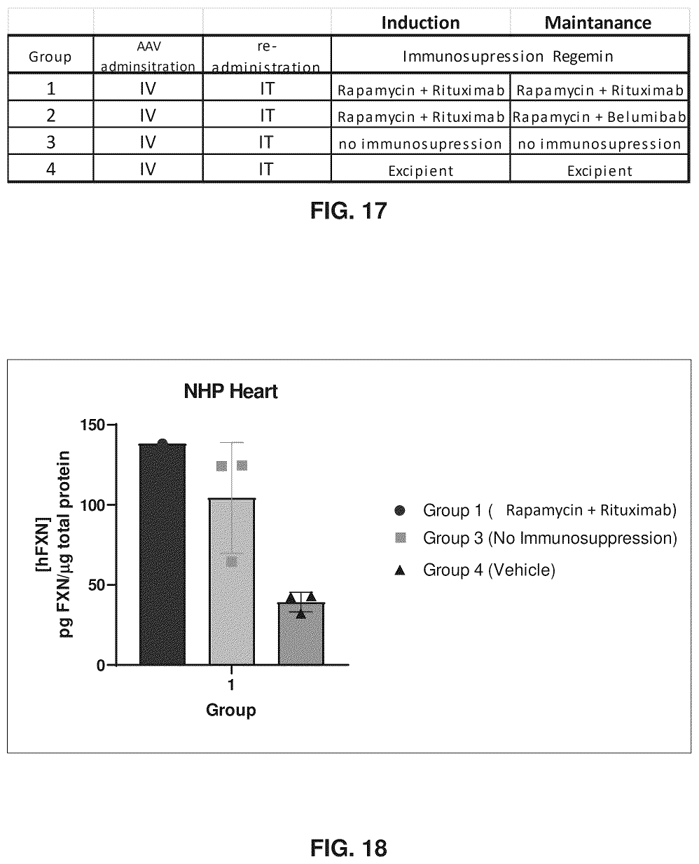

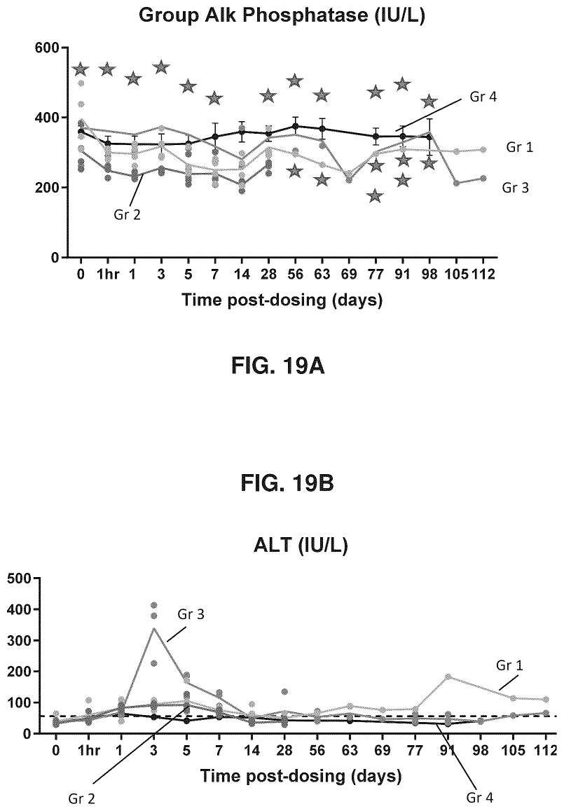

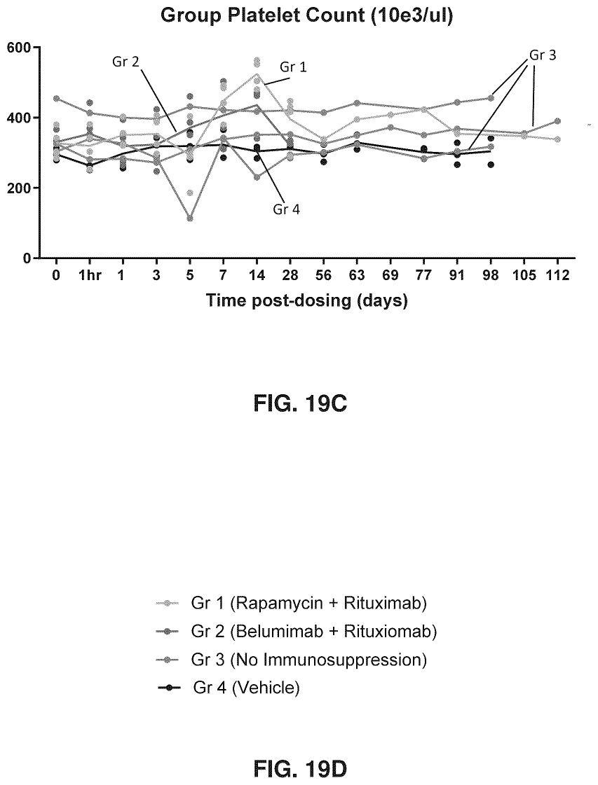

Non-human primates (NHP) were dosed as shown in FIG. 17. Alkaline phosphatase, alanine aminotransferase (ALT) and platelet levels were measured over time (FIGS. 19A-19D). The heat tissue was extracted from the NHPs and the level of frataxin protein was measured (FIG. 18).

### Phenotypic Mouse Model

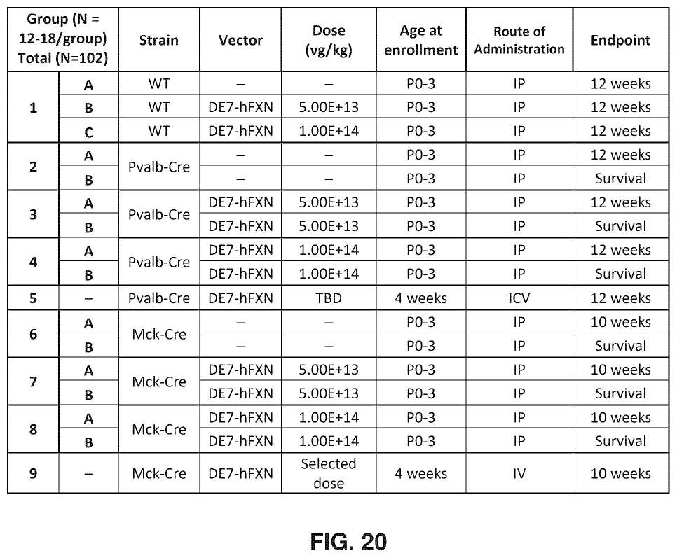

Wildtype and mutant mice are dosed as shown in FIG. 20 to assess the ability of DE7-hFXN to ameliorate the phenotype of Mck-CRE (muscle creatine kinase; muscle and heart targeting) and Pvalb-Cre mice (parvalbumin; neuron-targeting) that lack frataxin expression.

### OTHER EMBODIMENTS

All the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be illustrative examples, and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g, “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A recombinant adeno associated virus (rAAV) vector comprising a frataxin control region, exon 1 noncoding and coding region, frataxin intron, and frataxin exons 2-5.

2. The rAAV vector of claim 1, wherein the frataxin control region comprises a sequence that is at least 90, percent identical to any one of SEQ ID NOs: 3, 16, and 17.

3. The rAAV vector of claim 1, wherein the exon 1 noncoding region comprises a sequence that is at least 90, percent identical to SEQ ID NO: 4.

4. The rAAV vector of claim 1, wherein the exon 1 coding region comprises a sequence that is at least 90, percent identical to SEQ ID NO: 5.

5. The rAAV vector of claim 1, wherein the frataxin intron comprises a sequence that is at least 90, percent identical to SEQ ID NO: 6.

6. The rAAV vector of claim 1, wherein the frataxin exons 2-5 comprise a sequence that is at least 90, percent identical to SEQ ID NO: 7.

7. The rAAV vector of claim 1, wherein the rAAV vector encodes, from 5′ to 3′, the frataxin control region, the exon 1 noncoding region, the exon 1 coding region, the frataxin intron, and the frataxin exons 2-5.

8. The rAAV vector of claim 1, further comprising an additional element, wherein the additional element comprises an enhancer sequence, a polyA-encoding sequence, inverted terminal repeats (ITRs), or a 3′ untranslated region (UTR) sequence.

9. The rAAV vector of claim 1, wherein the rAAV vector comprises the sequences shown in Table 1.

10. The rAAV vector of claim 1, wherein the rAAV vector comprises the sequences shown in Table 2.

11. The rAAV vector of claim 1, wherein the rAAV vector comprises the sequences shown in Table 3.

12. The rAAV vector of claim 1, wherein the rAAV vector comprises the sequences shown in Table 4.

13. The rAAV vector of claim 1, wherein the rAAV vector comprises the sequences shown in Table 5.

14. An rAAV particle comprising the rAAV vector of claim 1.

15. A composition comprising the rAAV particle of claim 14.

16. The composition of claim 15, further comprising a pharmaceutically acceptable carrier.

17. A method comprising administering the rAAV particle of claim 14 to a subject.

18. A method of increasing frataxin expression in a cell, the method comprising administering an effective amount of the rAAV particle of claim 14 to the cell.

19. A method of treating Friedreich's Ataxia in a subject, the method comprising administering an effective amount of the rAAV vector of claim 1 to the subject.

20. The method of claim 17, wherein the subject is human.

21. The method of claim 20, wherein the subject has, is suspected of having, or is at risk for FRDA.

22. The method of claim 17, further comprising administration of one or more immunosuppressive compounds.

23. The method of claim 22, wherein the one or more immunosuppressive compounds comprise one or more of an anti-CD20 agent, Sirolimus, or Belimumab.
